(19) United States
(12) Patent Application Publication
(10) Pub. No.: US 2016/0089397 A1
(43) Pub. Date: Mar. 31, 2016

(54) CORRELATES OF EFFICACY RELATING TO TUMOR VACCINES

(71) Applicant: NEWLINK GENETICS CORPORATION, Ames, IA (US)

(72) Inventors: Gabriela ROSSI, Ames, IA (US); Charles LINK, Ames, IA (US)

(21) Appl. No.: 14/891,064

(22) PCT Filed: May 15, 2014
(86) PCT No.: PCT/US2014/038231

§ 371 Date: Nov. 13, 2015

Related U.S. Application Data
(60) Provisional application No. 61/823,873, filed on May 15, 2013.

(51) Int. Cl. A61K 35/13; C12N 13/00; C12N 9/10; C12N 5/09

(52) U.S. Cl. CPC A61K 35/13; C12N 5/0693; C12N 13/00; C12N 9/1051; C12N 2510/00; C12Y 204/01087

(57) ABSTRACT

The invention relates to methods and compositions for causing the selective targeting and killing of tumor cells. Through a combination of ex vivo gene therapy protocols and cell enchment, tumor cells are engineered to express an α (1,3)galactosyl epitope and optionally the tumor associated antigens mesothelin and carcinoembryonic antigen. After administration of the compositions of the invention to patients, the production of increased antibody titers to certain cell-surface markers, including mesothelin, calreticulin, and carcinembryonic antigen (CEA) positively correlates with an increased overall survival.

### FIELD OF THE INVENTION

The present invention relates to methods and compositions for treating cancer by stimulating humoral and cellular immune responses against tumor cells. In particular, this invention is directed to toward methods of producing improved whole cell tumor vaccines and identifying markers which correlate with improved patient outcome.

### BACKGROUND OF THE INVENTION

The basic rationale for immune therapy against tumors is the induction of an effective immune response against tumor-associated antigens (TAA), which in turn results in immune-mediated destruction of proliferating tumor cells expressing these antigens. For an immune response to be effective against TAAs comprising protein, these antigens must first be endocytosed by antigen presenting cells (APC) such as macrophages, dendritic cells and B cells. Within APCs, TAAs are degraded in the lysosomal compartment and the resulting peptides are expressed on the surface of the macrophage cell membrane mostly in association with MHC Class II molecules but also in association with MHC class I molecules. This expression mediates recognition by specific CD4⁺ helper T cells and subsequent activation of these cells to effect the immune response (Stevenson, 1991, FASEB J. 5:2250; Lanzavecchia, 1993, Science 260:937; Pardoll, 1993, Immunol. Today 14:310). The majority of human TAA molecules have not been defined in molecular terms, preventing these for use as targets for drug therapy or as anti-tumor vaccines.

As described in U.S. 2011/0250233, (herein incorporated by reference in its entirety) autologous and allogeneic tumor cells may be engineered to express an αGal epitope to induce an immune response which selectively targets and kills tumor cells. The engineered tumor cells are killed and/or attenuated (by gamma or ultraviolet irradiation, heat, formaldehyde and the like) and administered to a patient. The αGal epitope causes opsonization of the tumor cell which enhances tumor specific antigen presentation of antigens present in the entire tumor cell. The αGal epitope expressed on the surface of the modified cancer cell is important for processing of tumor associated antigens present within the entire tumor cell regardless of whether those proteins have been affected by the addition of αGal epitopes or not. Since αGal modifications affect multiple glycoproteins and glycolipids on the cell-surface, the patient's immune system will have an increased opportunity to detect, process, and generate antibodies to induce a cellular immune response to tumor specific antigens. The patient's immune system thus is stimulated to produce tumor specific antibodies and immune cells, which will attack and kill αGal negative tumor cells present in the animal that bear these tumor associated antigens.

### SUMMARY OF THE INVENTION

The present inventors have identified certain cell-surface markers expressed on the cell-surface of a tumor cell population modified to express αGal. After administration of these modified tumor cell populations to a patient, these cell-surface markers induce the production of antibodies, the levels of which correlate with an increased overall survival in patients. The present invention provides a tumor cell population modified to express αGal that also expresses mesothelin and carcinoembryonic antigen (CEA) on the cell-surface. After administration of these cells to cancer patients, the increased expression of antibodies directed to these markers correlates with an improved overall survival. The present invention provides a method of altering the immunotherapy dosage or adding other anti-cancer treatments to the treatment regimen depending on the antibody titers produced by the patient after administration of the compositions of the invention.

The present invention provides a method to produce a pancreatic antitumor composition effective in a patient comprising the steps of introducing into an isolated, non-tumorigenic cancer cell population a polynucleotide expression cassette having a functional α (1,3)-galactosyltransferase (αGT) protein, isolating and enriching for a transduced cancer cell population which expresses αGal, mesothelin and/or carcinoembryonic antigen on the cell-surface irradiating such cells. The present invention also provides the antitumor composition produced by this method.

The present invention provides a method to produce a pancreatic antitumor composition effective in a patient comprising the steps of introducing into an isolated, non-tumorigenic cancer cell population a polynucleotide expression cassette having a functional α (1,3)-galactosyltransferase (αGT) sequence, introducing into the modified cancer cell population one or more polynucleotide expression cassettes having a mesothelin and/or carcinoembryonic polynucleotide sequences, or fragments thereof, isolating a transduced cancer cell population which expresses αGal, mesothelin, and/or carcinoembryonic antigen on the cell-surface irradiating such cells. The present invention also provides the antitumor composition produced by this method.

In one embodiment, the invention provides an isolated, non-tumorigenic cancer cell population modified to express αGal, which also express mesothelin, calreticulin, and/or carcinoembryonic antigen (CEA) on the cell-surface, wherein after administration to a cancer patient, the production of antibodies to αGal, mesothelin, calreticulin, and/or carcinoembryonic antigen in said patient correlates with an improved overall survival. In another embodiment, the αGal expressed on the cell-surface is a trisaccharide of formula Galα1-3Galα1-4Glc, or Galα1-3Galα1-4GlcNAc. In another embodiment, the cancer cell is a pancreatic cancer cell. In a further embodiment, a least a 10-fold increase in anti-αGal antibodies compared to baseline correlates with improved overall survival. In a further embodiment, an increase in the levels of anti-mesothelin antibodies compared to baseline correlates with improved overall survival. In yet a further embodiment, an increase of about 25% or more of anti-mesothelin antibodies compared to baseline correlates with improved overall survival. In yet another embodiment, an increase in the levels of anti-carcinoembryonic antigen antibodies compared to baseline, correlates with improved overall survival. In yet another embodiment, an increase in the levels of anti-calreticulin antibodies compared to baseline correlates with improved overall survival. In yet a further embodiment, an increase of about 20% or more of anti-calreticulin antibodies compared with baseline correlates with improved overall survival.

In one embodiment, an increase in antibodies to one or more of αGal, mesothelin, calreticulin, and/or carcinoembryonic antigen in said patient correlates with an improved overall survival compared to that of patients exhibiting no increase in antibodies to any of these markers. In another embodiment, an increase in antibodies to two or more of αGal, mesothelin, calreticulin, and/or carcinoembryonic antigen in said patient correlates with an improved overall survival compared to that of patients exhibiting an increase in antibodies to one or two of these markers. In a further embodiment, an increase in antibodies to αGal, mesothelin, calreticulin, and carcinoembryonic antigen in said patient correlates with an improved overall survival compared to that of patients exhibiting an increase in antibodies to two or three of these markers.

In one embodiment, the compositions of the invention are administered in conjunction with one or more chemotherapeutic agents. In a further embodiment, the chemotherapeutic agent is gemcitabine. In another embodiment, the compositions of the invention are administered in conjunction with radiation therapy. In a further embodiment, the radiation therapy is 5FU chemo-radiation therapy. In another embodiment, the compositions of the invention are administered in conjunction with one or more chemotherapeutic agents and radiotherapy. In a further embodiment, the chemotherapeutic agent is gemcitabine and the radiation therapy is 5-FU chemo-radiation therapy.

### DESCRIPTION OF THE FIGURES

FIG. 1 shows the Schedule of Immunization for the NLG0205 pancreatic cancer clinical trials testing Algenpantucel-L (HyperAcute® Pancreas Immunotherapy). Patients enrolled in this Phase II pancreatic clinical trial received two immunizations of Algenpantucel-L before the first chemotherapy cycle after surgery. Subsequently, the patients received immunizations while receiving radiation therapy and/or chemotherapy. Serum samples were collected immediately before the first immunization to determine baseline levels. Serum samples were then obtained on Day 1 of cycle #2, Days 1 and 43 of chemoradiation, Day 1 of cycle #3, Day 1 of cycle #4, Day 1 of cycle #5, and at every follow-up visit.

FIG. 2 shows the accuracy and precision of the ELISA method used in these studies. Two operators performed this study using 4 reference normal pool sera (NPS) samples (NPS7-NPS8, NPS9 and NPS10) and the reference standard. The percent coefficient of variation (CV) or Relative Standard Deviation (RSD) within and between experiments is expected to be ≦20% and accuracy is suggested to be in the range of 80-120%.

FIG. 3 shows the determination by ELISA of anti-αGal antibody values in reference samples by two operators in multiple experiments.

FIG. 4 shows the anti-αGal antibody titers of patients before receiving Algenpantucel-L immunotherapy. The baseline value of anti-αGal antibodies varies significantly among patients. Patients tested in this trial had a mean titer of 24 μg/mL with a range of 2 to 149 μg/ml.

FIG. 5 is a graph comparing the levels of α-Gal antibodies found in patient sera before immunization with Algenpantucel-L and overall survival. There is no apparent correlation with baseline antibody values and survival (p=0.1074) indicating that there is no apparent predictive value for the amount of anti-αGal antibodies produced in a patient before immunization and a better prognosis or outcome.

FIGS. 6A-6G show the levels of anti-α-Gal antibodies detected in all tested patients after immunization with Algenpantucel-L. After immunization the vast majority of tested patients responded by increasing the levels anti-αGal antibodies produced. Of 50 patients tested, 46 (92%) responded with at least 2-fold increased anti-αGal antibody levels compared to pre-immunization values. The level of the response varied significantly among patients.

FIG. 7 shows the increase in anti-α-Gal antibody levels in patients following immunization. The mean fold-response (test/baseline) for the entire NLG0205 trial showed a 16 fold increase in anti-αGal antibody levels (range 2 to 128) compared to baseline. Patients receiving 300M cells tend to exhibit a higher anti-αGal antibody response compared to patients receiving 100M dose cells. Patients in the 300M dose cohort have a mean fold-increase of 23 compared to 13 in the 100M dose cohort. These data suggest a dose-response in the induction of anti-αGal antibodies in these patients.

FIG. 8 shows that there is a statistically significant correlation between the development of high titers of anti-αGal antibodies and better outcome (Overall Survival) in 50 tested patients.

FIG. 9 shows that the correlation of increased anti-α-Gal antibody production in patients with better outcome (Overall Survival) is observed only in the group of patients receiving high doses (300M) of Algenpantucel-L.

FIG. 10 shows there is no correlation between overall survival and anti-αGal antibody levels observed in patients in a clinical trial testing Tenrgenpumatucel-L (HyperAcute® Lung Immunotherapy) in lung cancer patients. Consequently the data observed on the pancreatic trials is unique to the pancreatic trial.

FIG. 11 shows the performance characteristic of the control sample NPS10. NPS10 was tested in each experiment on each plate as a control. Testing of NPS10 was consistent with less than 10% CV for the determination of both the slope and the y-intercept, demonstrating that the values obtained during this study have acceptable quality with variation among experiments within acceptable range.

FIG. 12 shows the obtained upper limit of quantification (ULOQ) values obtained for the anti-αGal antibody standard and the acceptable range of expected values (dotted lines). The ULOQ values observed were within acceptable range and the % CV observed was less than 10% indicating acceptable degree of variability.

FIG. 13 shows the accuracy and precision of the ELISA method used in these studies. The values obtained for all the experiments and the summary for the performance of the anti-αGal antibody standard. The variability and coefficient determinates are within the expected and acceptable range.

FIG. 14 shows that several of the cell lines tested that are components of HyperAcute-Pancreas and Lung vaccines express high levels of CEA tumor associated antigen. CEA RNA can be detected in HAL1, HAL3, HAPa1, BxPC3 and Capan 2 cells.

FIG. 15 shows that 17 out of 63 patients enrolled in NLG0205 showed a statistically significant increase in the anti-CEA antibody levels post-immunization with Algenpantucel-L. This clustering of an anti-CEA antibody response is characterized by a threshold of 20% increase in the response after immunization compared to baseline. This clustering of response was statistically significant and potentially clinically meaningful (p<0.0001).

FIG. 16 is a graph showing the survival of patients producing or not producing increased anti-CEA antibody levels after immunization with Algenpantucel-L. Patients that seroconverted to higher levels of anti-CEA antibody levels after immunizations showed improved overall survival compared to patients with no increase in anti-CEA antibody levels.

FIG. 17 shows that there is a not a statistically significant correlation between the titer of anti-CEA antibodies produced in the patient after immunization and better outcome, indicating that the response itself (sero-conversion) and not the magnitude of the response is associated with better outcome.

FIG. 18 shows that the administered dose of Algenpantucel-L does not affect the percent change in the levels of anti-CEA antibodies produced in the patient following immunization. There is no difference in the change in anti-CEA antibody levels observed in patients receiving who received 300M of Algenpantucel-L compared with those who received the 100M of Algenpantucel-L, suggesting that at least concerning the anti-tumor immune response measured by the change in the levels of this antibody, both dose regimes seem similar.

FIG. 19 shows the preliminary analysis of the overall survival of patients analyzed in a lung cancer study. The production of anti-CEA antibodies in patients after administration of Tenrgenpumatucel-L (HyperAcute® Lung Immunotherapy) does not positively correlate with increased overall survival of patients.

FIG. 20 shows the performance characteristic of the control sample NPS10. NPS10 was tested in each experiment on each plate as a control. Testing of NPS10 was consistent with less than 10% CV for the determination of both the slope and the y-intercept, demonstrating that the values obtained during this study have acceptable quality with variation among experiments within acceptable range.

FIG. 21 shows the detection by RT-PCR of mesothelin RNA in pancreatic cell lines. One of the cell line components of Algenpantucel-L immunotherapy, HAPa1, expresses detectable levels of mesothelin antigen.

FIG. 22 shows that membrane-bound mesothelin can be detected by FACS analysis in pancreatic cell lines. HAPa1 cells possess membrane-bound mesothelin on the cell-surface. An ovarian cancer cell line (CaoV3) that shows high expression of mesothelin was used as a positive control.

FIG. 23 shows the levels of anti-mesothelin antibody (anti-MSLN) produced in patients after immunization. A clustering of anti-mesothelin antibody response is characterized by a threshold of a 25% increase in the anti-MSLN antibody titers after immunization compared to baseline. Of the 64 patients evaluated, 20 (31%) patients showed an increased anti-MSLN antibody response after immunization.

FIG. 24 shows the sub-group analysis of patients producing or not producing elevated anti-MSLN antibody levels following immunization. Patients who seroconverted to anti-MSLN antibodies had a better outcome, with a median overall survival of 42 months compared to patients who had no anti-MSLN antibody response after immunization and had a median overall survival of 20 months.

FIG. 25 shows the correlation of elevated anti-MSLN antibody levels and overall survival in immunized patients. There is a statistically significant correlation between the development of anti-MSLN antibodies and better outcome.

FIG. 26 shows the performance characteristic of the control sample NPS10 for these studies. NPS10 was tested in each experiment on each plate as a control. Testing of NPS10 was consistent with less than 10% CV for the determination of both the slope and the y-intercept, demonstrating that the values obtained during this study have acceptable quality with variation among experiments within acceptable range.

FIG. 27 shows the survival analysis of pancreatic cancer patients after receiving Algenpantucel-L. There are three groups of patients: those who exhibited no increase in antibody response, those who exhibited an increase in one type of antibody production, and those who exhibited an increase in the production of two or more antibodies. An increased antibody titer in a patient after immunization correlates with a better overall survival (p=0.012). Patients responding with one type of antibody studied (n=26) had a significantly better outcome compared to patients with no antibody response (n=27) after immunization (26 months vs. 17 months, p=0.047). Patients responding with two or more types of antibodies had an even better outcome—as of Jan. 23, 2013, the median level of survival has not yet been reached.

FIG. 28 shows the comparison of median survival including the confidence intervals of the three groups of patients. The likelihood of a patient's responding to therapy is significantly greater if an increase in antibody titer is observed after immunization with Algenpantucel-L. Those patients who showed no antibody response (n=27) showed a survival rate of 19%, while those showing an increased level of one antibody after immunization (n=26) have a survival rate of 42% (p=0.0476). Those patients showing an increased level of two or more antibodies studied (n=13) have a survival rate of 69% (p=0.0073).

FIG. 29 shows an increase in eosinophil levels after immunization with Algenpantucel-L. Patients that show an increase in eosinophil levels at least three times during the course of immunization have a median survival of 27 months compared to a median survival of 21 months in those patients who did not exhibit an increase in eosinophil levels.

FIG. 30 shows skin biopsies which indicate presence of eosinophils at the injection sites might be unique to Algenpantucel-L.

FIG. 31A-D shows different receptors present on dying cells. Panel A represents lytic/necrotic death, panel B represents apoptotic death, panel C represents apoptotic cells expressing clareticulin on their surface, and panel D represents cell markers that stimulate phagocytosis.

FIG. 32 shows the expression of calreticulin on both HAPa1 and HAPa2 cells.

FIG. 33 shows the clustering of anti-calreticulin antibody response post-immunization with Algenpantucel-L.

FIG. 34 shows the Kaplan-Meir graph of the sub-group analysis of patients responding or not with elevated anti-CALR antibodies after immunization with Algenpantucel-L.

FIG. 35 shows the reactivity of NPS10 anti-CALR, anti-CEA, and anti-Mesothelin in a qulaified normal pool ser sample (NPS10) detected by Western blot.

FIG. 36 shows the variability in the detection of NPS10 reactivity against calreticulin intra-experiment. The variability for the uppler limit of detection of anti-CALR antibodies present in NPS10 is below 10% in all experiments except EXP03, where the variability observed was 17.66%.

FIG. 37 shows the variability observed inter-experiment. Triangles denote the mean value expected plus or minus 1.75 standard deviations (SD); squares denote the mean value of all experiments; circles denot the values obtained.

FIG. 38 shows the serial dilution curve for NPS10 and their corresponding OD value.

FIG. 39 shows a linear regression of the inter-experiment variability. This figure shows the average valued for each point with error bars as SD. The solid line with no circles represents the fitted curve.

### DETAILED DESCRIPTION OF THE INVENTION

Cancer immunotherapy is an emerging form of cancer treatment in which the patient is administered with an engineered tumor cell to induce an immune response against the cancer cells, thereby targeting the pre-existing tumor for destruction. Some forms of immunotherapy use allogeneic tumor cells genetically engineered to express αGal epitopes on the cell-surface. These cells are estimated to contain at least one to two million αGal epitopes (U.S. 2011/0250233, herein incorporated by reference in its entirety). This large number of binding sites for naturally pre-existing anti-αGal antibody results in a high density of opsonization followed by complement destruction which sets off a variety of processes that activate both the humoral and cellular branches of the immune system. The presence of such a high density of αGal residues on the surface of allogeneic tumor cells induces a hyperimmune response analogous to xenograft hyperacute rejection at the site of the modified tumor cell injection. Furthermore, these cancer vaccines are polyvalent meaning that they present multiple tumor antigen targets to the immune system. This will result in a more efficient treatment in that several TAAs will be presented and in a more widely effective treatment as with the increased number of TAAs presented it is more likely that there will be overlap in epitopes from different individual tumors. Opsonized cells are readily ingested by phagocytes providing a mechanism whereby most of the tumor antigens can be simultaneously presented to the adaptive immune system. Within these cells, proteins from the cancer vaccine cells will be digested and given class II MHC presentation thereby exposing the mutant proteins epitopes in the cancer cell to T-cell surveillance. In addition, the uptake of opsonized cells by antigen presenting cells (APCs) via Fc receptor mediated endocytosis may facilitate the activation of MHC class I restricted responses by CD8⁺ cells through a cross presentation pathway. The immune system cascade set in motion by this process provides the stimulus to induce a specific T-cell response to destroy native tumor cells from an established human malignancy. Furthermore, the inflammatory environment induced by the primary immune response results in an amplification effect mediated by cytokines, histamines and other up-regulated molecules that boost the T-cell response. T-cells activated in this manner are directly capable of killing cancer cells. The addition of αGal epitopes to glycoproteins and glycolipids present in the tumor vaccine will not restrict the development of an immune response only to those antigens that become glycosylated but to any antigen present within the tumor cell whether it is affected by glycosylation or not.

Natural anti-αGal antibodies are of polyclonal nature and synthesized by 1% of circulating B cells. They are present in serum and human secretions and are represented by IgM, IgG and IgA classes. The main epitope recognized by these antibodies is the αGal epitope (Galα1-3Galβ1-4NAcGlc-R) but they can also recognize other carbohydrates of similar structures such as Galα1-3Galβ1-4Glc-R, Galα1-3Galβ1-4NAcGlcβ1-3Galβ1-4Glcβ.-R, Galα1-3Glc (melibiose), α-methyl galactoside, Galα1-6Galα1-6Glcβ (1-2)Fru (stachyose), Galα1-3(Fucα1-2)Gal-R (Blood B type epitope), Galα1-3Gal and Galα1-3Gal-R (Galili et al. 1987; Galili et al. 1985; Galili et al. 1984). Similarly, non-natural synthetic analogs of the αGal epitope have been described to bind anti-αGal antibodies and their use has been proposed to deplete natural anti-αGal antibodies from human sera in order to prevent rejection of xenogeneic transplants (Janczuk et al. 2002; Wang et al. 1999). Therefore, glycomimetic analogs of the αGal epitope could also be used to promote the in vivo formation of immunocomplexes for vaccination purposes. Other carbohydrates such as rhamnose and Forssman antigen may also be used (U.S. application Ser. No. 13/463,420 herein incorporated by reference in its entirety).

Applicants' invention provides the identification of cell-surface markers which, when enriched on a population of engineered tumor cells that express αGal epitopes or other suitable carbohydrates, induce the production of antibodies in the patient that positively correlate with an increased overall survival.

The compositions of the invention comprise tumor cells that are engineered to express a αGal epitopes (or other suitable carbohydrate epitopes). Such epitopes may be added by expressing in the cells a nucleic acid encoding an alpha galactosyltransferase (αGT) or other suitable enzyme, for example a viral or non-viral vector. Alternatively, such epitopes may be inserted directly into the cell membrane or conjugated to proteins on the cell surface. These modified cells are enriched for the presence of certain cell-surface markers, including, but not limited to, mesothelin, calreticulin, and/or carcinoembryonic antigen (CEA), and are then lethally irradiated or otherwise killed and administered to a patient. The binding of αGal epitopes by naturally pre-existing anti-αGal antibodies causes opsonization of the tumor cells and enhances tumor specific antigen presentation. The invention contemplates the use of whole cells, and a mixture of a plurality of transduced cells in the pharmaceutical compositions of the invention. Since αGal modifications affect multiple glycoproteins on the cell-surface, the patient's immune system will have an increased opportunity to detect, process, and generate antibodies to tumor specific antigens.

One embodiment of the invention comprises transfection of tumor cells with a nucleotide sequence which encodes upon expression, the enzyme α-(1,3)-galactosyl transferase (αGT). The αGT cDNA has been cloned from bovine and murine cDNA libraries. Larson, R. D. et al. (1989) “Isolation of a cDNA Encoding Murine UDP galactose; β-D-galactosyl-1,4-N Acetol-D-Glucosamine α1-3. Galactosyl Transferase: Expression Cloning by Gene Transfer”, PNAS, USA 86:8227; and Joziasse, D. H. et al., (1989) “Bovine α1-3 Galactosyl Transferase: Isolation and Characterization of a cDNA Clone, Identification of Homologous Sequences in Human Genomic DNA”, J. Biol Chem 264:14290. Any other nucleotide sequence which similarly will result in the tumor cells expressing an αGal epitope on the cell-surface may be used according to the invention, for example other enzymes that catalyze this reaction or perhaps event the engineering of the cells to have additional glycoproteins present on the cell-surface hence the artificial creation of a TAA which can be presented to the immune system.

The tumor cells of the present invention may be syngeneic, allogeneic, or autologous. The transformed cells and the tumor cells to be treated must have at least one epitope in common, but will preferable have many. To the extent that universal, or overlapping epitopes or TAA exist between different cancers, the pharmaceutical compositions may be quite widely applicable.

Applicants have surprisingly found that after immunization with compositions of the invention, the production of antibodies in a patient to certain cell-surface markers positively correlates with an increased overall survival. The data described herein demonstrate that the levels of antibodies to αGal, mesothelin, calreticulin, and/or CEA produced by the patient after immunization with a composition of the invention correlate with an increased overall survival for the patient. In one embodiment, at least a 10-fold increase in anti-αGal antibodies after immunization with a composition of the invention correlates with an increased overall survival for the patient.

Overall survival for a patient has also been found to correlate with the number of cell-surface markers to which the patient demonstrates an increase in antibody titers. Applicants have found that patients who produce elevated antibody titers to αGal, mesothelin, calreticulin, or CEA after administration of immunotherapy have a better overall survival than patients who do not produce elevated antibody titers to any of these antigens. Additionally, those patients who produce elevated titers to two or more of these antigens after administration of immunotherapy have a better overall survival than those who produce antibodies to only one or two of these antigens.

One aspect of the present invention provides for an isolated, non-tumorigenic tumor cell population which has been modified to express αGal and also expresses mesothelin, calreticulin, and/or CEA. The expression of mesothelin, calreticulin, and/or CEA on the surface of these modified cells may be achieved through any standard means in the art, including, but not limited to enrichment of the cell population by selecting for those cells which already express one or both of these antigens, or by engineering a cell through recombinant means to express one or both of these antigens.

Use of traditional techniques for cell sorting, such as by immunoselection (including, but not limited to, FACS), permits identification, isolation, and/or enrichment for αGal(+) cells that express mesothelin, calreticulin, and/or CEA. The reagent can be an anti-mesothelin antibody, an anti-CEA antibody, an anti-calreticulin antibody, an anti-αGal antibody or a combination thereof. The modified tumor cells expressing αGal are grown in culture and in one embodiment, FACS is used to select those αGal(+) cells from the population expressing mesothelin, calreticulin, and/or CEA. The selection step can further entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference in all its entirety). The αGal expressing tumor cells that are found to express mesothelin and/or CEA are then cultured further and expanded.

Alternatively, the modified αGal-expressing tumor cell can be recombinantly engineered to express mesothelin, calreticulin, and/or CEA. Using standard techniques known in the art, polynucleotides encoding these antigens or fragments thereof can be inserted into the modified tumor cell for expression of one or more of these antigens on the cell surface. In one embodiment, the modified αGal expressing tumor cell is transduced with an expression vector comprising a mesothelin polynucleotide or fragment thereof for expression of the mesothelin antigen on the cell. In another embodiment, the modified αGal-expressing tumor cell is transduced with an expression vector comprising a CEA polynucleotide or fragment thereof for expression of the CEA antigen on the cell. In another embodiment, the modified αGal-expressing tumor cell is transduced with an expression vector comprising a calreticulin polynucleotide or fragment thereof for expression of the calreticulin antigen on the cell. In another embodiment, the modified αGal-expressing tumor cell is transduced with an expression vector comprising a mesothelin polynucleotide or fragment thereof for expression of the mesothelin antigen on the cell, and an expression vector comprising a CEA polynucleotide or fragment thereof for expression of mesothelin and CEA on the cell-surface. In another embodiment, the modified αGal-expressing tumor cell is transduced with an expression vector comprising polynucleotide sequences of both mesothelin and CEA or fragments thereof for expression of these antigens on the cell. In another embodiment, the modified αGal-expressing tumor cell is transduced with an expression vector comprising a calreticulin polynucleotide or fragment thereof for expression of calreticulin on the cell surface and an expression vector comprising a mesothelin polynucleotide or fragment thereof and/or an expression vector comprising a CEA polynucleotide or fragment thereof for expression of calreticulin and mesothelin and/or CEA on the cell-surface. In a further embodiment, the modified αGal-expressing tumor cell is transduced with an expression vector comprising polynucleotide sequences of calreticulin and mesothelin and/or CEA or fragments thereof for expression of these antigens on the cell.

The αGalactosyltransferase, mesothelin, calreticulin, and/or CEA nucleic acid sequences or fragments thereof, can be contained in an appropriate expression vehicle which transduces tumor cells. Such expression vehicles include, but are not limited to, eukaryotic vectors, prokaryotic vectors (for example, bacterial vectors), and viral vectors. In one embodiment, the expression vector is a viral vector. Viral vectors that may be employed include, but are not limited to, retroviral vectors, adenovirus vectors, herpes virus vectors, and adeno-associated virus vectors, or DNA conjugates. Examples of retroviral vectors which may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus.

The vector includes one or more promoters. Suitable promoters which may be employed include, but are not limited to, the retroviral LTR, the SV40 promoter, and the human cytomegalovirus (CMV) promoter described in Miller, et al. Biotechniques, Vol. 7, No. 9, 980-990 (1989) (incorporated herein by reference in its entirety), or any other promoter (e.g. cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters.

In another embodiment the invention comprises an inducible promoter. One such promoter is the tetracycline-controlled transactivator (tTA)-responsive promoter (tet system), a prokaryotic inducible promoter system which has been adapted for use in mammalian cells. The tet system was organized within a retroviral vector so that high levels of constitutively-produced tTA mRNA function not only for production of tTA protein but also the decreased basal expression of the response unit by antisense inhibition. See, Paulus, W. et al., “Self-Contained, Tetracycline-Regulated Retroviral Vector System for Gene Delivery to Mammalian Cells”, J of Virology, January. 1996, Vol. 70, No. 1, pp. 62-67. The selection of a suitable promoter will be apparent to those skilled in the art from the teachings contained herein.

The vector then is employed to transduce a packaging cell line to form a producer cell line. Examples of packaging cells which may be transfected include, but are not limited to the PE501, PA317, ¥2, ¥-AM, PA12, T19-14X, VT-19-17-H2, ¥-CRE, ¥-CRIP, GP+E-86, GP+envAM12, DAN and AMIZ cell lines. The vector containing the nucleic acid sequence encoding the agent which is capable of providing for the destruction of the tumor cells upon expression of the nucleic acid sequence encoding the agent, and activation of the complement cascade may transduce the packaging cells through any means known in the art. Such means include, but are not limited to, electroporation, the use of liposomes, and CaPO₄ precipitation. In one embodiment, the invention comprises a viral vector which commonly infects humans and a packaging cell line which is human based. For example vectors derived from viruses which commonly infect humans such as Herpes Virus, Epstein Barr Virus, may be used.

After administration of the compounds of the invention to patients, the antibody levels to αGal, mesothelin, calreticulin, and/or CEA in the patient samples may be measured by immunoassays commonly used in the art (see, e.g., Harlow & Lane, Antibodies, A Laboratory Manual (1988), hereby incorporated by reference in its entirety, for a description of immunoassay formats and conditions that can be used to determine specific immunoreactivity). Non-limiting examples of such assays include, but are not limited to, radioimmunoassay, indirect immunofluorescence assays (IFA), and ELISA. Suitable immunoassay methods typically include: receiving or obtaining (e.g., from a patient) a sample of body fluid or tissue likely to contain antibodies; contacting (e.g., incubating or reacting) a sample to be assayed with an antigen, under conditions effective for the formation of a specific antigen-antibody complex (e.g., for specific binding of the antigen to the antibody); and assaying the contacted (reacted) sample for the presence of an antibody-antigen reaction (e.g., determining the amount of an antibody-antigen complex). The presence of an elevated amount of the antibody-antigen complex indicates that the subject has produced antibodies to the marker on the cell-surface. An antigen, including a modified form thereof, which “binds specifically” to (e.g., “is specific for” or binds “preferentially” to) an antibody against a cell surface marker interacts with the antibody, or forms or undergoes a physical association with it, in an amount and fora sufficient time to allow detection of the antibody. By “specifically” or “preferentially,” it is meant that the antigen has a higher affinity (e.g., a higher degree of selectivity) for such an antibody than for other antibodies in a sample. For example, the antigen can have an affinity for the antibody of at least about 1.5-fold, 2-fold, 2.5-fold, 3-fold, or higher than for other antibodies in the sample. Such affinity or degree of specificity can be determined by a variety of routine procedures, including, e.g., competitive binding studies. In an ELISA assay, a positive response is defined as a value 2 or 3 standard deviations greater than the mean value of a group of unimmunized controls. Phrases such as “sample containing an antibody” or “detecting an antibody in a sample” are not meant to exclude samples or determinations (e.g., detection attempts) where no antibody is contained or detected. In a general sense, this invention involves assays to determine whether an antibody produced in response to immunization with compositions of the invention is present in a sample, irrespective of whether or not it is detected.

In one embodiment, the measurement of antibody levels in the patient samples is accomplished by ELISA. In one embodiment, the reagents for evaluating antibody expression are polypeptide antigens. In another embodiment, the antigen is αGal. In a further embodiment, the antigen is CEA. In yet a further embodiment, the antigen is mesothelin. The levels of one or more of these antibodies in the patient sample may be measured using one or more of these reagents.

Patient samples that may be tested for the levels of antibodies to αGal, mesothelin, calreticulin, and/or CEA produced by patients after administration of the compositions of the invention include, but are not limited to, blood, plasma, and/or serum. In one embodiment, the patient sample is serum.

The measurement of antibody titers to αGal, mesothelin, calreticulin, and/or CEA may be useful for the early identification of patient populations who will or will not benefit from treatment with the compositions of the invention. The measurement of the levels of antibody titers to certain cell-surface markers may be used to maintain current treatment, change the course or dosage of treatment, or add alternate therapies. Patients may respond to immunotherapy by producing increased antibodies to zero, one, two, or all three of these antigens. In one embodiment, patients who produce increased antibodies to none or one of these antigens are given an increased dosage of the compositions of the invention or put on additional forms of cancer therapy, including but not limited to, IDO inhibitors, chemotherapy, alternate immunotherapy, radiation, and/or a combination thereof.

The antibodies produced by the patient to the cell-surface molecules may be measured after one, two, three, four, five, six, seven, eight, nine, ten, or more immunizations with the compounds of the invention. In one embodiment, the antibodies produced by the patient to the cell-surface molecules are measured after two immunizations with the compounds of the invention. In a further embodiment, the antibodies produced by the patient to the cell-surface molecules are measured after five immunizations with the compounds of the invention. In yet a further embodiment, the antibodies produced by the patient to the cell-surface molecules are measured after ten immunizations with the compounds of the invention.

In one embodiment, the invention provides a method of treating cancer or an uncontrolled cellular growth comprising administering the compounds of the invention. Tumors which may be treated in accordance with the present invention include malignant and non-malignant tumors. Cells from malignant (including primary and metastatic) tumors include, but are not limited to, those occurring in the adrenal glands; bladder; bone; breast; cervix; endocrine glands (including thyroid glands, the pituitary gland, and the pancreas); colon; rectum; heart; hematopoietic tissue; kidney; liver; lung; muscle; nervous system; brain; eye; oral cavity; pharynx; larynx; ovaries; penis; prostate; skin (including melanoma); testicles; thymus; and uterus. Examples of such tumors include apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, Merkel cell, mucinous, non-small cell lung, oat cell, papillary, scirrhous, bronchiolar, bronchogenic, squamous cell, and transitional cell), plasmacytoma, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myoma, myosarcoma, rhabdomyoma, rhabdomyosarcoma, ependymoma, ganglioncuroma, glioma, mcdulloblastoma, meningioma, neurilemnnoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyorna, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (e.g., Ewing's experimental, Kaposi's, and mast-cell), neoplasms and for other such cells.

In one embodiment, a patient may demonstrate an increase in eosinophil levels after administration with the compounds of the invention which correlates with an increased overall survival. In one embodiment, an increase of eosinophil levels at least three times after administration of the compounds of the invention correlates with an increased overall survival.

In one embodiment, both increased production of eosinophils and antibodies to αGal, mesothelin, calreticulin, and/or CEA in a patient are measured after administration with the compounds of the invention. In another embodiment, a patient who demonstrates a lack of an increase in eosinophils and/or antibody titer to one or more of these antigens is given a higher dose of the compounds of the invention or put on additional forms of cancer therapy, including but not limited to, IDO inhibitors, chemotherapy, alternate immunotherapy, radiation, and/or a combination thereof.

According to the invention, attenuated αGal expressing tumor cells enriched for the expression of mesothelin, calreticulin, and/or CEA are used as either prophylactic or therapeutic vaccines to treat tumors. Thus the invention also includes pharmaceutical preparations for humans and animals involving these transgenic tumor cells. Those skilled in the medical arts will readily appreciate that the doses and schedules of pharmaceutical composition will vary depending on the age, health, sex, size and weight of the human and animal. These parameters can be determined for each system by well-established procedures and analysis e.g., in phase I, II and III clinical trials and by review of the examples provided herein.

The compositions of the invention are generally administered in therapeutically effective amounts. The term “therapeutically effective amount” is meant an amount of treatment composition sufficient to elicit a measurable decrease in the number, quality or replication of previously existing tumor cells as measurable by techniques including but not limited to those described herein. These compositions may be administered in a single dose or in multiple doses. Standard dose-response studies, first in animal models and then in clinical testing, reveal optimal dosages for particular disease states and patient populations. In some embodiments, an effective dosage of the vaccine of the invention will contain at least 100 million or more cells. In another embodiment, an effective dosage will comprise at least about 300 million or more cells. In another embodiment, an effective dosage will comprise at least about 500 million or more cells.

For administration, the compositions of the invention can be combined with a pharmaceutically acceptable carrier such as a suitable liquid vehicle or excipient and an optional auxiliary additive or additives. The liquid vehicles and excipients are conventional and are commercially available. Illustrative thereof are distilled water, physiological saline, aqueous solutions of dextrose, and the like.

Suitable formulations for parenteral, subcutaneous, intradermal, intramuscular, oral, or intraperitoneal administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example ethyl oleate or triglycerides. Aqueous injection suspensions may contain substances which increase the viscosity of the suspension, include for example, sodium carboxymethyl cellulose, sorbitol, and/or dextran. Optionally, the suspensions may also contain stabilizers. Also, compositions can be mixed with immune adjuvants well known in the art such as Freund's complete adjuvant, inorganic salts such as zinc chloride, calcium phosphate, aluminum hydroxide, aluminum phosphate, saponins, polymers, lipids or lipid fractions (Lipid A, monophosphoryl lipid A), modified oligonucleotides, etc.

In addition to administration with conventional carriers, active ingredients may be administered by a variety of specialized delivery drug techniques which are known to those of skill in the art.

For administration, the modified tumor cells can be combined with a pharmaceutically acceptable carrier such as a suitable liquid vehicle or excipient and an optional auxiliary additive or additives. The liquid vehicles and excipients are conventional and are commercially available. Illustrative thereof are distilled water, physiological saline, aqueous solutions of dextrose and the like.

The compositions of the invention can be administered alone or in conjunction with other cancer treatments. In one embodiment, the compositions of the invention may be administered in conjunction with chemotherapeutic agents. In another embodiment, the compositions of the invention may be administered in conjunction with radiation therapy. In yet a further embodiment of the invention, the compositions of the invention may be administered in conjunction with one or more chemotherapeutic agents and radiation therapy.

Examples of chemotherapeutic agents which may be administered in conjunction with the compositions of the invention include, but are not limited to, alkylating agents, such as nitrogen mustards (e.g., mechlorethamine, cyclophosphamide, ifosfamide, melphalan, and chlorambucil); nitrosoureas (e.g., carmustine (BCNU), lomustine (CCNU), and semustine (methyl-CCNU)); ethyleneimines and methyl-melamines (e.g., triethylenemelamine (TEM), triethylene thiophosphoramide (thiotepa), and hexamethylmelamine (HMM, altretamine)); alkyl sulfonates (e.g., buslfan); and triazines (e.g., dacabazine (DTIC)); antimetabolites, such as folic acid analogues (e.g., methotrexate, trimetrexate, and pemetrexed (multi-targeted antifolate)); pyrimidine analogues (such as 5-fluorouracil (5-FU), fluorodeoxyuridine, gemcitabine, cytosine arabinoside (AraC, cytarabine), 5-azacytidine, and 2,2′-difluorodeoxycytidine); and purine analogues (e.g, 6-mercaptopurine, 6-thioguanine, azathioprine, 2′-deoxycoformycin (pentostatin), erythrohydroxynonyladenine (EHNA), fludarabine phosphate, 2-chlorodeoxyadenosine (cladribine, 2-CdA)); Type I topoisomerase inhibitors such as camptothecin (CPT), topotecan, and irinotecan; natural products, such as epipodophylotoxins (e.g., etoposide and teniposide); and vinca alkaloids (e.g., vinblastine, vincristine, and vinorelbine); anti-tumor antibiotics such as actinomycin D, doxorubicin, and bleomycin; radiosensitizers such as 5-bromodeozyuridine, 5-iododeoxyuridine, and bromodeoxycytidine; platinum coordination complexes such as cisplatin, carboplatin, and oxaliplatin; substituted ureas, such as hydroxyurea; and methylhydrazine derivatives such as N-methylhydrazine (MIH) and procarbazine; and inhibitors of microtubule function such as docetaxel and paclitaxel. In one embodiment, the chemotherapeutic agent is gemcitabine. The chemotherapeutic agent administered in combination with the compositions of the invention is administered as determined by the treating physician, and at doses typically given to patients being treated for cancer.

Examples of radiation therapy that may be administered in conjunction with compositions of the invention include, but are not limited to, radiation emitters such as alpha-particle emitting radionuclides (e.g., actinium and thorium radionuclides), low linear energy transfer (LET) radiation emitters (i.e. beta emitters), conversion electron emitters (e.g. strontium-89 and samarium-153-EDTMP, or high-energy radiation, including without limitation x-rays, gamma rays, and neutrons. The radiation therapy may be performed with a sensitizer, including but not limited to, 5FU. The radiation therapy administered in combination with the compositions of the invention is administered as determined by the treating physician, and at doses typically given to patients being treated for cancer.

The compositions of the invention and the further therapeutic agent may be given simultaneously in the same formulation. Alternatively, the agents are administered in a separate formulation but concurrently, with concurrently referring to agents given, for example, within minutes, hours or days of each other. In some embodiments, the compositions of the invention comprise a plurality of autologous tumor cells which may be the same or different. The autologous tumor cells may be administered separately or together.

In another aspect, the further therapeutic agent is administered prior to administration of the compositions of the invention. Prior administration refers to administration of the further therapeutic agent within the range of minutes, hours, or one week prior to treatment with the compositions of the invention. It is further contemplated that the further therapeutic agent is administered subsequent to administration of the compositions of the invention. Subsequent administration is meant to describe administration more than minutes, hours, or weeks after administration of the compositions of the invention.

The present invention also provides a kit for the detection of antibodies produced to the αGal, mesothelin, calreticulin, and/or CEA in a patient receiving immunotherapy. In a non-limiting example, one or more reagents for evaluating antibody expression can be provided in a kit. In one embodiment, the kit contains one reagent to measure the expression levels of one antibody in the patient sample. In another embodiment, the kit contains the reagents to measure the expression of two antibodies the patient sample. In yet a further embodiment, the kit contains the reagents to measure the levels of three antibodies in the patient sample. In a further embodiment, the kit contains the reagents to measure the levels of more than three antibodies in a patient sample. The kits may thus comprise, in suitable container means, nucleic acids, antibodies, polypeptides, or other regents that can be used to determine antibody titers in a sample. In one embodiment, the reagents are attached or fixed to a support, such as a plate, chip or other non-reactive substance. For example, a reagent can be fixed to a microtiter well, and the sample placed in the well to determine the expression level of antibodies to the cell-surface markers expressed on the compounds of the invention. In one embodiment, the reagents for evaluating antibody expression are polypeptide antigens. In another embodiment, the antigen is αGal. In a further embodiment, the antigen is CEA. In yet a further embodiment, the antigen is mesothelin. In yet a further embodiment, the antigen is calreticulin.

The kits may comprise a suitably aliquoted nucleic acids that can be used as probes or primers; alternatively, it may comprise a suitably aliquoted antibody that can be used in immunohistochemical detection methods or any other method discussed herein or known to those of skill in the art.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the containers in close confinement for commercial sale. Such means may include injection or blow-molded plastic containers into which the desired vials are retained.

To confidently and immediately measure the levels of antibody production in patients receiving immunotherapy with the compounds of the invention, assays to measure the antibody titers may be performed at the point of care using transportable, portable, and handheld instruments and test kits. Small bench analyzers or fixed equipment can also be used when a handheld device is not available. In one embodiment of the invention, a kit is provided to the point-of-care to allow immediate testing of the levels of anti-cell-surface markers produced in patients receiving immunotherapy.

### EXAMPLES

The following examples are provided to further illustrate the advantages and features of the invention, but are not intended to limit the scope of this disclosure. All citations to patents and journal articles are hereby expressly incorporated by reference in their entireties.

### Example 1

### Humoral Immunologic Response Before and After Immunization With Algenpantucel-L (HyperAcute-Pancreas NLG0205).

We evaluated whether in patients enrolled in Phase II clinical trials vaccination with Alegenpantucel-L HyperAcute-Pancreas immunotherapy induced antibodies against αGAL epitopes, the cell-surface marker Carcinoembryonic antigen (CEA) and the recombinant membrane bound mesothelin (MSLN).

Patients enrolled in the Phase II pancreatic clinical trials NLG0205 received two immunizations before the first chemotherapy cycle after surgery. Subsequently, they received immunizations while receiving radiation therapy and/or chemotherapy.

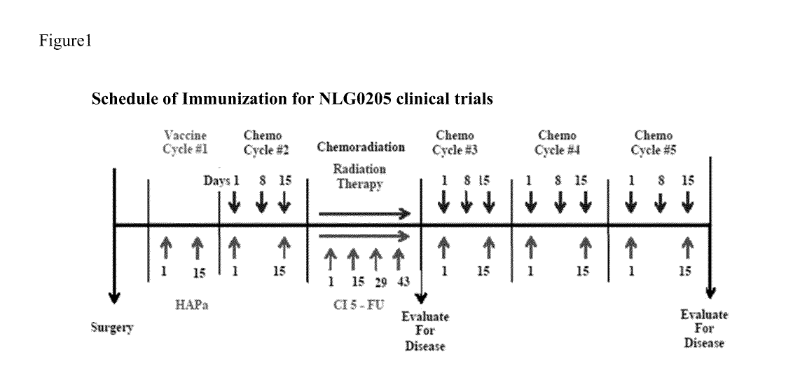

Serum samples for the study of the humoral immune response (anti-αGal antibody, anti-CEA antibody and anti-MSLN antibody) were collected immediately before the first immunization to determine the baseline values. Serum samples were obtained on Day 1 of cycle #2, days 1 and 43 of chemoradiation, Day 1 of cycle #3, Day 1 of cycle #4, Day 1 of cycle #5, and at every follow-up visit (FIG. 1).

### Assay Qualification/Validation for the Detection of Anti-αGal Antibodies Before and After Immunization in Serum Samples by ELISA.

Measurements of immune response provide important potential surrogate endpoints for monitoring the efficacy of anti-cancer immunotherapy trials. Anti-αGalactosyl antibody Enzyme-Linked Immunosorbent Assay (anti-αGal antibody ELISA) is an endpoint assay used in immunological studies on human serum samples obtained from clinical trials of NewLink Genetics cancer immunotherapy (HyperActute). In this study we improved and characterized the performance quality and consistency of the assay to demonstrate that it is a suitable and reliable method for quantifying the anti-αGal antibodies in serum samples from clinical trial. This summary report provides a description of the anti-αGal antibody ELISA method validation and the performance characteristics of the assay that were established. The study was based mainly on method validation guidelines published by the US Food and Drug Administration (FDA), 2001 and the ICH Harmonized Tripartite Guidelines, 2005.

### Brief Assay Description

The detection of anti-αGal antibodies was performed by ELISA. Briefly A 96-well microliter plate is coated with α-Gal-HSA (antigen) overnight, washed and blocked with HSA at 37° C. Samples (Primary Antibodies) are dispensed on the plate, allowed to react with antigen and washed. Enzyme conjugated secondary antibodies are dispensed on the plate and allowed to react with primary antibodies. A chromogenic detection substrate is dispensed on plate and allowed to react with conjugate yielding a product with blue color. Reaction is stopped with 2M Sulphuric acid and optical density (OD) of samples is detected with a plate reader at a wavelength of 450 nm. Analysis of data is performed using Microsoft Excel and/or GraphPad Prism software. In each plate the purified standard is tested in duplicates wells, a qualified normal pooled serum sample (NPS10) is also tested in each experiment as an additional quality control reagent. All patients samples are tested in each plate.

### Optimized Anti-αGal ELISA Parameters

Accurate validation of a bioanalytical procedure is only possible when the operational parameters or conditions of the method are optimized. In this regard, several experiments were performed to determine the optimal conditions for the anti-αGal ELISA method. The validation parameters of the method and procedural efficiency within and between experiments and operators established herein presumes optimized assay conditions and stable assay reagents and samples. Only under such conditions can the performance characteristics of the method presented in this report be expected to be reproducible within limits of random experimental error.

### Results and Conclusions

The established parameters for optimal anti-αGal antibody ELISA method are as presented in Table 1. These assay conditions constitute the elements of the Standard Operation Procedure (SOP) for application and validation of the anti-αGal antibodies ELISA method as presented herein. The validation parameters established herein are therefore only applicable under the optimized conditions presented below, changes to which may necessitate partial re-validation of the method or otherwise:

TABLE 1
Optimized assay conditions for anti-αGal ELISA protocol.

| Reagent | Concentration/time | |
|---|---|---|
| Antigen (αGal-HSA) in carbonate- | 5 | μg/ml |
| bicarbonate buffer | | |
| Antigen (αGal-HSA) coating per well (50 μl) | 250 | ng |
| Coating buffer (HSA) in carbonate- | 1% | |
| bicarbonate buffer | | |
| Secondary antibody (Goat anti-Human | 1/24000 | |
| IgG-HRP conjugated Ab) | | |
| TMB substrate concentration | 0.4 | mg/ml |
| Hydrogen peroxide concentration (in citric | 0.02% | |
| acid buffer) | | |
| TMB substrate-Hydrogen peroxidase mix | 100 | μl/well |
| Stopping reagent (100 ml/well) | 2M H₂SO₄ | |
| Substrate incubation time | 20 | minutes |

### Calibration of Standard Curve

Calibration of the anti-αGal antibody standard curve is the empirical determination of the relationship between measured absorbance (OD) values for the Standards and the true or known concentration of the standards. A chromatographically purified total human IgG was further affinity purified to obtain the anti-αGal IgG standard used in this study The Limit of Detection (LOD), Lower Limit of Quantification (LLOQ), and Upper Limit of Quantification (ULOQ) are essential values that define or delimit the dynamic range of the standard curve. The range of the calibrated standard curve include a blank (matrix sample processed without internal standard), zero-sample (matrix sample processed with internal standard), and six non-zero sample points including the LLOQ and ULOQ.

Table 2 summarizes the findings described above.

TABLE 2
Performance characteristics of the anti-αGal antibody ELISA standard

| Performance characteristic | Actual/set values | |
|---|---|---|
| Limit of Detection (LOD)/Sensitivity | 1 | ng/ml |
| Lower Limit of Quantification (LLOQ) | 2 | ng/ml |
| Upper Limit of Quantification (ULOQ) | 20 | ng/ml |
| Dynamic range of standard curve | 0-20 | ng/ml |

| | |
|---|---|
| Standards (ng) | 0, .05, 2, 4, 8, |
| | 12, 16, 20 |
| Matrix effect | None |
| Selectivity/Recovery in serum | Acceptable: 90-110% |
| Dilution linearity | Acceptable: 90-110% |

### Stability of Assay Reagents

Consistency in experimental results is also greatly influenced by stability of reagents used in an assay. The stability of samples and reagents used in anti-αGal ELISA method were tested under conditions at which they are stored or processed in order to determine the time frame for stability of the samples and reagents. The storage conditions investigated are refrigeration at 4° C., and freezing at −20° C. or −80° C. for both samples and reagents. Exposure of reagents and samples to room temperature (RT) as well freeze-thaw of reagents or samples was investigated.

### Stability of the Standard Conclusions

The standard is stable on storage at 4° C. for at least up to 100 as indicated by a consistent or stable range of the OD values demonstrated for the standard with acceptable range of variability (ULOQ, % CV: 7.5 and LLOQ % CV 16%).

Freeze-thaw cycles affect the performance of the Standard. Data indicates a high coefficient of variation (CV: 35%) in experiments performed. Consequently, freezing and thawing is not recommended. A paired t-test comparison between 4° C. and RT exposure data indicate that the reactivity of the standard is not affected by exposure to room temperature for up to 4 hours.

### Stability of the Secondary Antibody Conclusions

Results show no significant change in performance of secondary antibody on exposure to room temperature for up to 72 hours (P=0.7868). A significant difference was observed when all data was compiled together indicating a slight trend for reduced performance of the secondary antibodies when exposed at RT for 192 hours. Consequently the secondary antibody is considered stable for at least 72 hours when stored at room temperature. The results also indicate that the secondary antibody is stable when stored at 4C for up to 18 months.

### Stability of the Samples

Patient's samples with low, intermediate and high titer of anti-αGal antibodies were tested for stability on storage at 4° C., exposure to room temperature for up to 4 hours, and freeze-thaw cycles.

Results show that freshly thawed and continuously monitored samples stored at 4° C. remain stable for up to 6 months. Results of exposure of sample to room temperature for up to 4 hours show no significant effect on estimate of analyte, and freeze-thaw cycles of samples stored at −20° C. did not show a specific trend indicating an adverse effect of freeze-thaw cycles on the samples. The results suggest that the samples are stable on freeze-thaw for at least up to 10 cycles.

### Procedural Efficiency, Precision and Accuracy.

The procedural efficiency is a measure of the application of the anti-αGal antibody ELISA methods in terms of accuracy and precision of data within and between experiments and operators. Under the optimized assay conditions, the percent coefficient of variation (CV) or Relative Standard Deviation (RSD) within and between experiments is expected to be ≦20% and accuracy is suggested to be in the range of 80-120%. The results for procedural efficiency obtained from analysis of reference NPS samples are presented below.

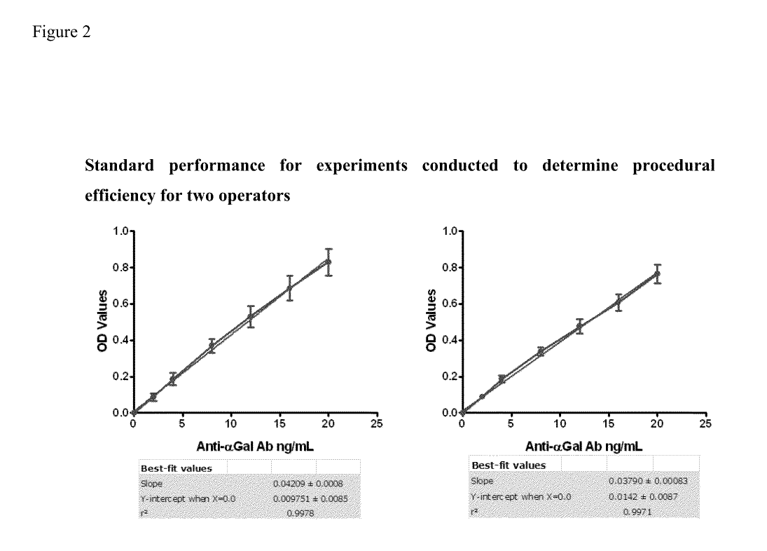

The procedure for testing anti-αGal antibodies was repeated multiple times to determine the precision and the accuracy of the method. Two operators performed this study using 4 reference normal pool sera (NPS) samples (NPS7-NPS8, NPS9 and NPS10) and the reference standard. A summary of the standard performance by two operators is shown in FIG. 2.

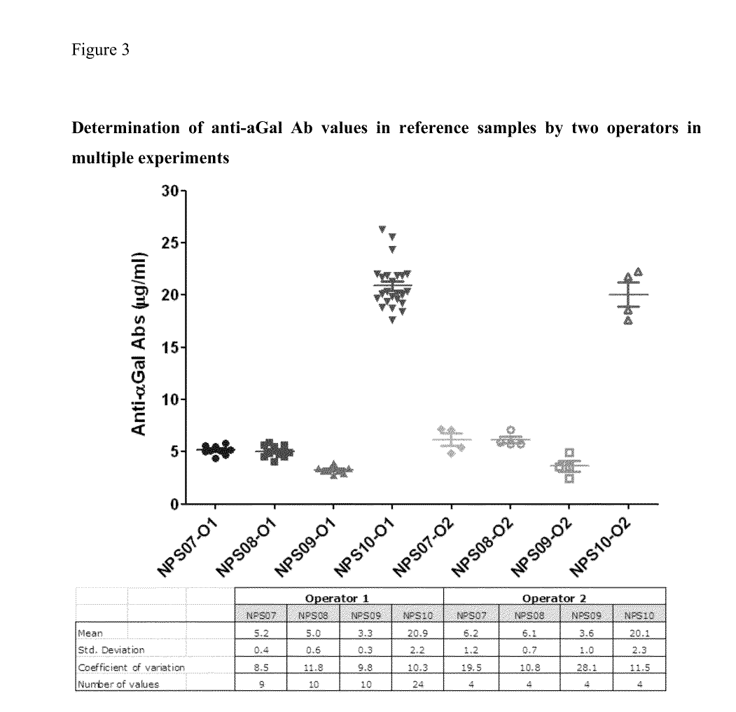

The estimates of anti-αGAL antibody values for reference samples obtained in these experiments are shown in FIG. 3.

The summary of the precision and accuracy is depicted in Table 3.

TABLE 3
Precision and Accuracy over experiments

| | Statistics | | | | |
|---|---|---|---|---|---|
| | Mean | SD | % CV | % ACC | n |
| Standard | 1007.42 | 31.43 | 3.12 | 100.74209 | 33 |
| NPS07 | 5.43 | 0.83 | 15.3 | | 31 |
| NPS08 | 5.29 | 0.80 | 15.1 | | 33 |
| NPS09 | 3.38 | 0.57 | 16.9 | | 33 |
| NPS10 | 21.21 | 2.75 | 13.0 | | 59 |

### Robustness

The robustness of an analytical procedure is the property that indicates insensitivity against changes made to known operational parameters on the results of the method which provide an indication of its suitability or reliability for its defined purpose. Insensitivity of a method to inadvertent changes made to known operational parameters between operators or laboratories defines ruggedness of the procedure.This report presents data on empirical investigation on the robustness of anti-αGal ELISA method for seven assay parameters that were selected and considered to be the most important in the procedure. The parameters in question are quantity of antigen (αGal-HSA) coated on plate, incubation time for primary antibody, incubation time for secondary antibody, wash cycles, substrate incubation time, TMB temperature, and time lapse before reading of plates. A Plackett-Burman design for screening of many variables or factors for their main effect (Plackett and Burman, 1964) is chosen for the study on the robustness of anti-αGal ELISA method. Table 4 shows the parameters evaluated during this study and results.

TABLE 4
Experimental factors and levels

| Factor | Factor description | −1 | | +1 | |
|---|---|---|---|---|---|
| X₁ | Coating with αGal-HSA per well | 235 | ng | 265 | ng |
| X₂ | Incubation time with primary antibody | 45 | min | 75 | min |
| X₃ | Incubation time with secondary antibody | 45 | min | 75 | min |
| X₄ | Wash cycles | 3 | cycles | 7 | cycles |
| X₅ | Substrate incubation time | 15 | min | 25 | min |
| X₆ | Temperature of substrate (TMB) | 4° C. | | 25° C. | |
| X₇ | Time lapse to reading of plate | 1 | min | 10 | min |

### Conclusions of the Validation/Qualification Study

This study demonstrates that the anti-αGal ELISA method is robust, shows acceptable precision and accuracy, and therefore suitable for quantification of anti-αGal antibody in patient serum samples.

### Example 2

### Detection of Anti-αGal Antibodies Before and After Immunization in Serum Samples by ELISA.

The detection of anti-αGal antibodies was performed by ELISA using standard techniques. Each experiment was considered valid if the following criteria for valid test were demonstrated.

### Criteria for Valid Test

Anti-αGal concentration of 21±5 μg/ml for NPS10 as analyte control.

OD value range for standard: Upper limit from 0.65 to 0.91.

Standard curve coefficient of determination (R2) ranging from 0.9800 to 0.9999.

### Results

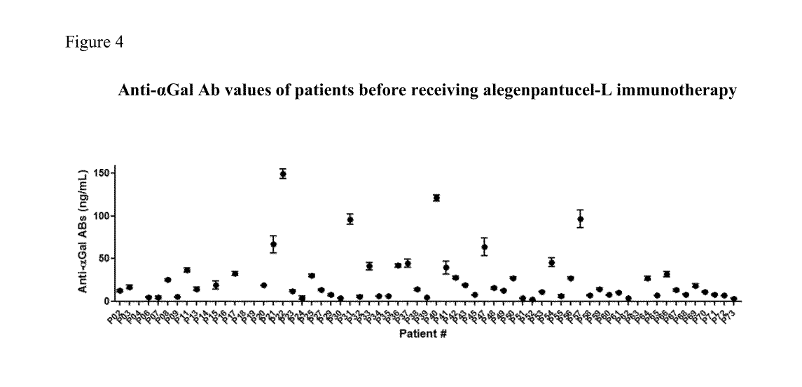

All patients tested in this trial had detectable anti-αGal antibodies before receiving Algenpantucel-L immunotherapy (FIG. 4).

As demonstrated in other clinical trials using HyperAcute technology the baseline values of anti-αGal antibodies varies significantly among patients. Patients tested in this trial had a mean antibody titer of 24 μg/mL with a range of 2 to 149 μg/ml.

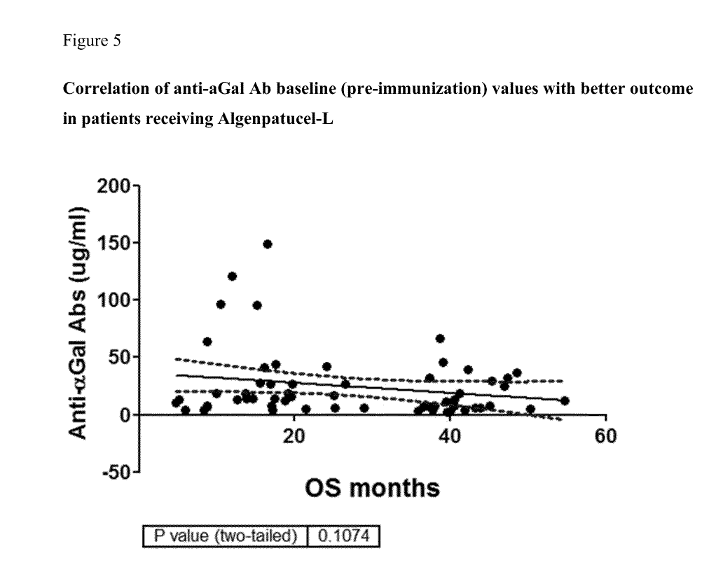

We performed a Pearson correlation study to determine if the baseline values of anti-αGal antibodies have any predictive value to a favorable survival. Data below shows that there is no apparent correlation with baseline values and survival (p=0.1074) indicating no apparent predictive value for the amount of anti-αGal antibodies before immunization and better prognosis or outcome (FIG. 5).

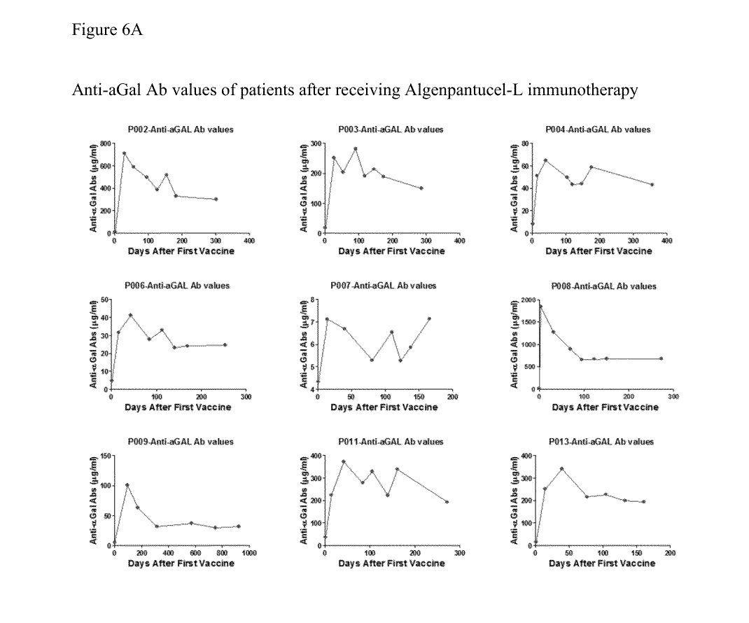

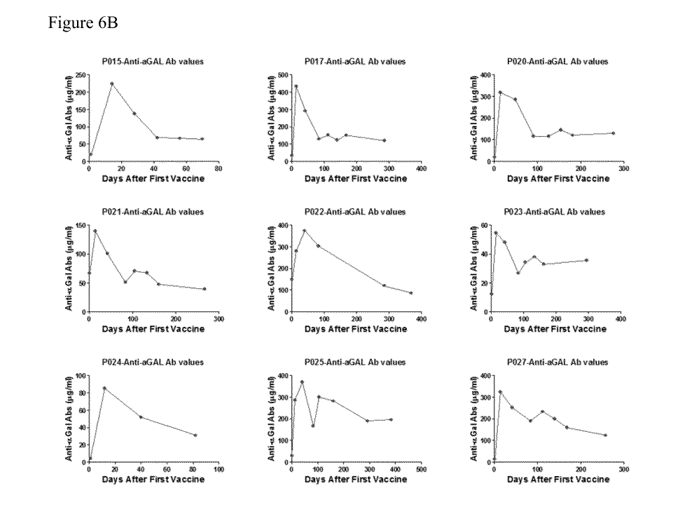

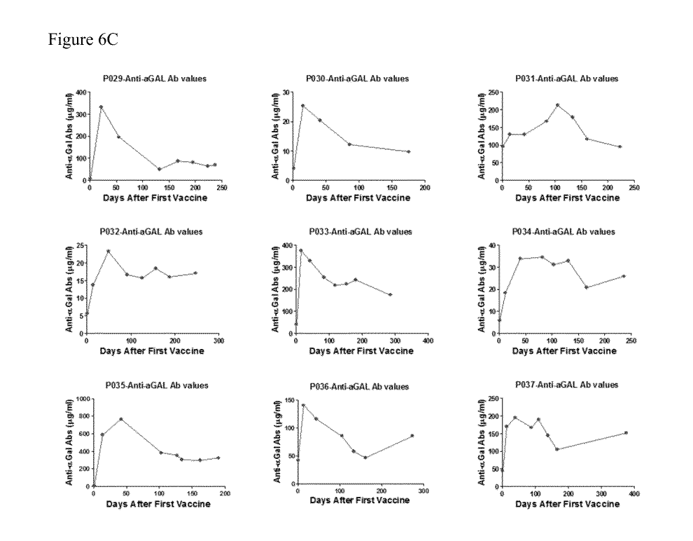

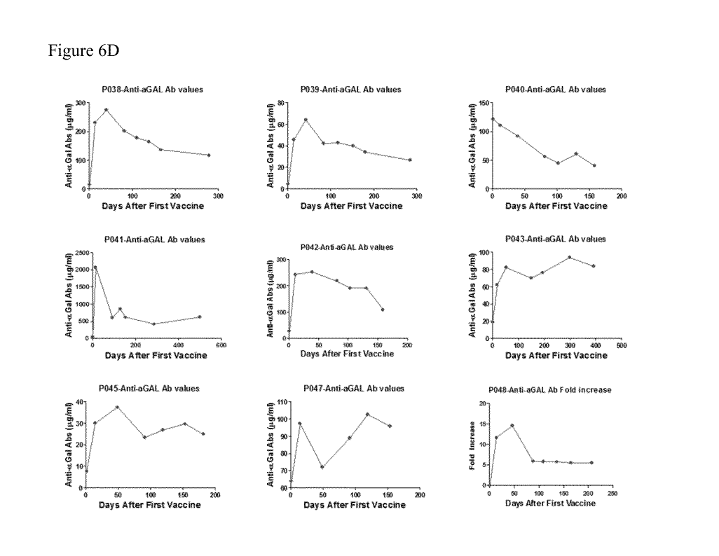

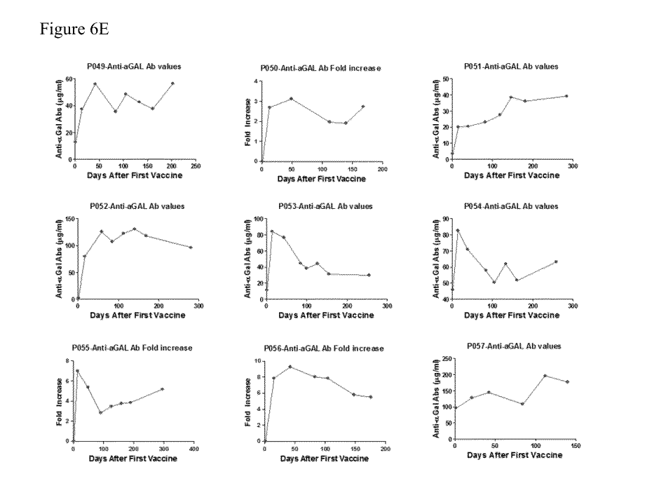

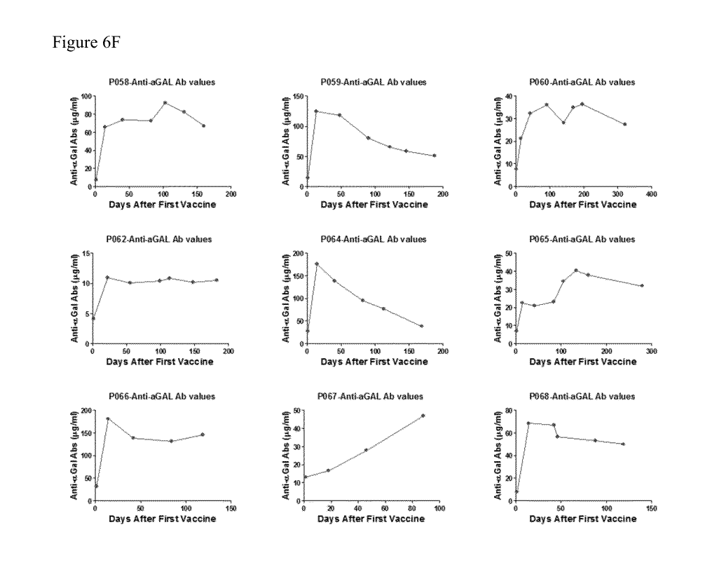

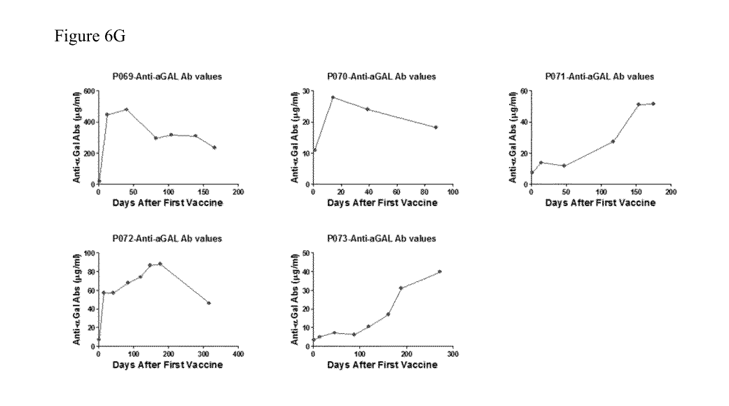

After immunization the vast majority of tested patients responded by increasing their anti-αGal antibody levels. In this study we tested 50 patients and 46 (92%) responded with at least 2-fold increase in the levels of anti-αGal antibodies compared to pre-immunization values. The level of the response varied significantly among patients. FIGS. 6A-6G show the levels of anti-αGal antibodies detected in all tested patients.

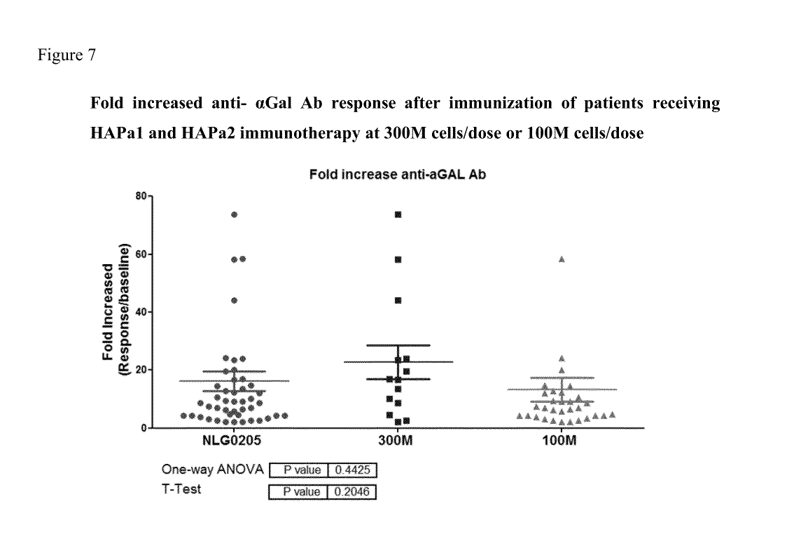

The magnitude of the response after vaccination was calculated by the fold-increase in the anti-αGal antibody response after immunization. The fold-increase is calculated by the ratio of the peak response divided by the baseline value. As shown in FIG. 7, the mean fold-response (test/baseline) for the entire NLG0205 trial was a 16 fold increase in anti-αGal antibody levels (range 2 to 128) compared to baseline. We performed the analysis comparing both dose cohort for the anti-αGal antibody response after immunization. As shown in FIG. 7, patients receiving 300M cells tend to have a higher anti-αGal antibody response compared to those patients receiving 100M dose cells. Patients in the 300M dose cohort have a mean fold-increase of 23 compared to 13 in the 100M dose cohort. These data suggest a dose-response in the induction of anti-αGal antibody response in these tested patients.

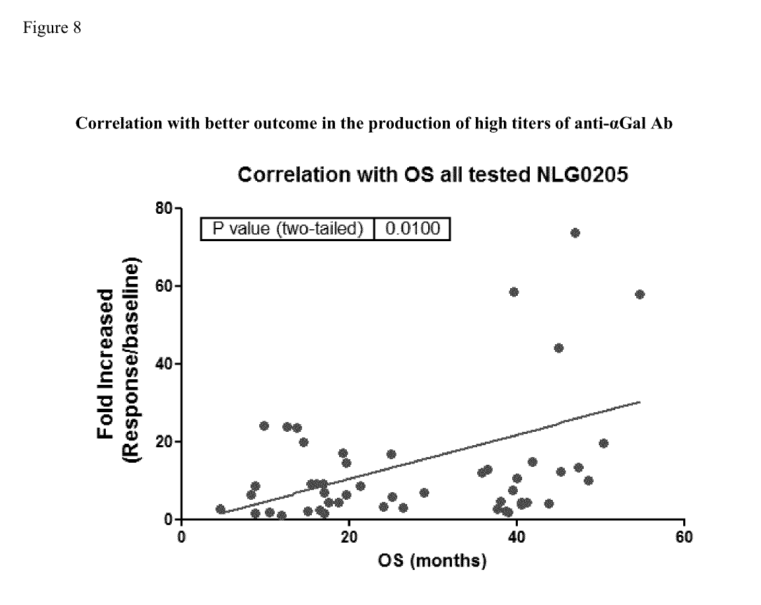

To determine if the magnitude of the response in anti-αGal antibody had a correlation with better outcome, we performed a Pearson correlation calculation. FIG. 8 demonstrates that there is a statistically significant correlation between the development of high titers of anti-αGal antibodies and better overall survival in 50 tested patients.

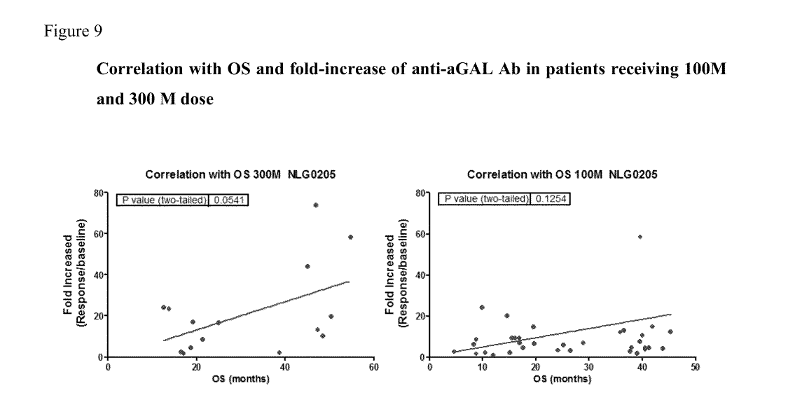

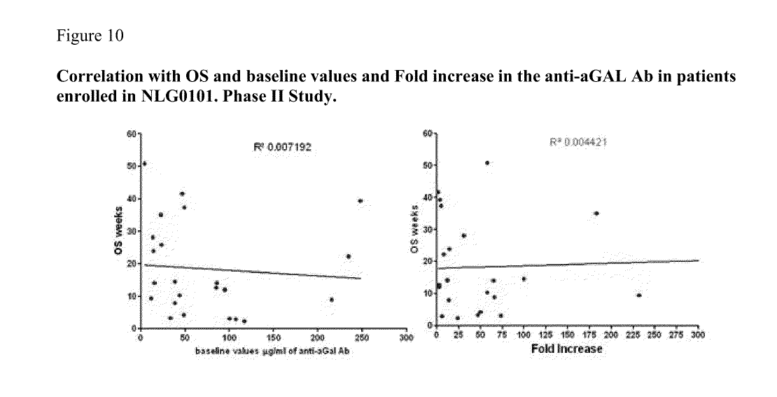

The correlation among favorable overall survival (OS) and increased anti-αGal antibody titers was performed for patients receiving 100M dose and 300M dose. As shown in FIG. 9, the correlation with better outcome is observed only in the group of patients receiving high doses on Algenpantucel-L suggesting again a dose-response. Furthermore, in trials of other cancers treated with different HyperAcute immunotherapy, the presence of increased titers of anti-αGal antibodies does not correlate with an improved overall survival. FIG. 10 shows there is no correlation between overall survival and anti-αGal antibody levels observed in patients in a clinical trial testing Tenrgenpumatucel-L (HyperAcute® Lung Immunotherapy) in lung cancer patients. While patients responded to therapy with increased anti-αGal antibody levels in the lung cancer study, the fold increase in anti-αGal antibody levels does not correlate with an increase in overall survival in patients. The correlation between the fold increase in anti-αGal antibody levels and overall survival is unique to the pancreatic trial.

### Conclusions

All patients had detectable pre-existing naturally acquired anti-αGal antibodies (mean 24 μg/mL, range 2 to 149 μg/ml). The vast majority of patients receiving Algenpantucel-L immunotherapy responded by increasing the anti-αGal antibody after immunization. In this study 46 out of 50 patients (92%) had increased anti-αGal antibody titer by at least 2 fold. The development of high titters of anti-αGal antibody correlated with better outcome for the entire clinical trail (p=0.01). Subgroup analysis indicated that in the 300M dose cohort correlation between anti-αGAL antibody response and better outcome was still observed in comparison to the 100M dose cohort where statistically significant correlation was lost. This result indicates that 300M dose cohort induced higher titers of anti-αGAL antibodies that correlated with better overall survival suggesting a dose-response effect.

### Example 3

### Performance Characteristic of the Anti-αGal Antibody ELISA Assay During the Testing of Patients Enrolled in NLG0205

The following section describes the assay performance during the testing of anti-αGal antibody values by ELISA.

As explained above, the criteria for valid test for this assay were developed during the qualification/validation process of this assay. It is expected that the value obtained for the NPS10 control sample will be 21±5 μg/ml, the upper limit of OD value for the standard curve should be with 0.65 to 0.91 OD units and the coefficient of determination for the standard curve is expected to be 0.9800 to 0.9999.

### Testing of NPS10

As explained above, each patient sample was analyzed in a single plate, In addition to patient's samples, we added a qualified commercially available reagent from normal pooled sera (NPS10). This reagent was tested extensively and it was established that the expected concentration of anti-αGal antibodies was 21±5 μg/ml.

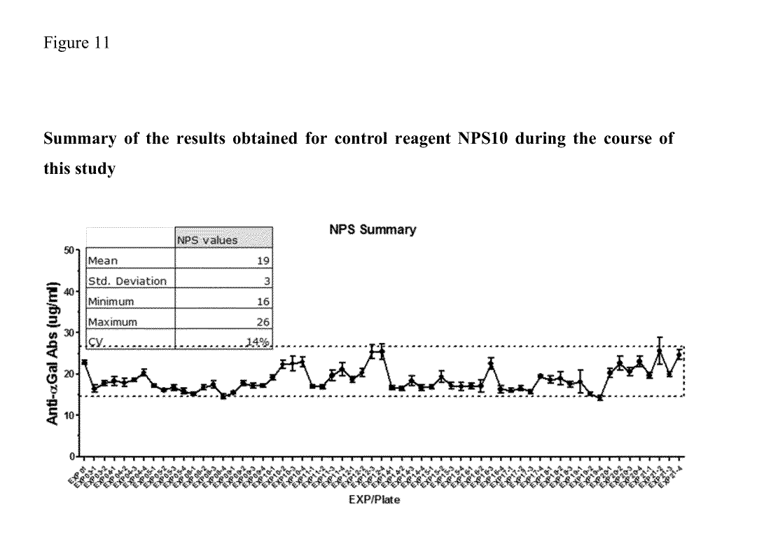

FIG. 11 shows the summary of values obtained during the course of this study. The expected variability is shown (dotted lines). As shown in FIG. 11, the percent coefficient of variability (% CV) in less than 15% indicating that variability observed in this study is within acceptable range.

### Purified Anti-αGal Antibody Standard Performance

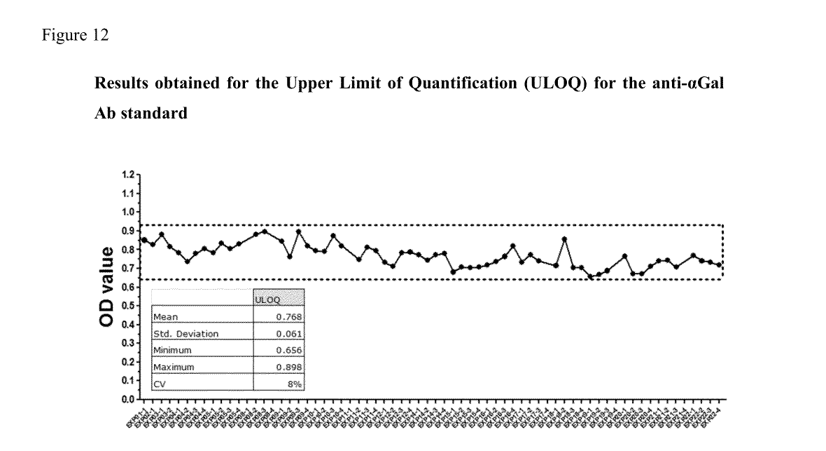

In order to quantify the amount of anti-αGal antibody values in patient's samples we utilized an affinity purified anti-αGal antibody standard. This reagent was fully characterized during the qualification/validation portion of this study. In the patients testing phase the anti-αGal antibody standard was included in each plate for each patient tested. FIG. 12 shows the obtained Upper limit of quantification (ULOQ) values obtained for the anti-αGal antibody standard and the acceptable range of expected values (dotted lines). As shown in FIG. 12, the ULOQ values observed were within acceptable range and the % CV observed was less than 10% indicating acceptable degree of variability.

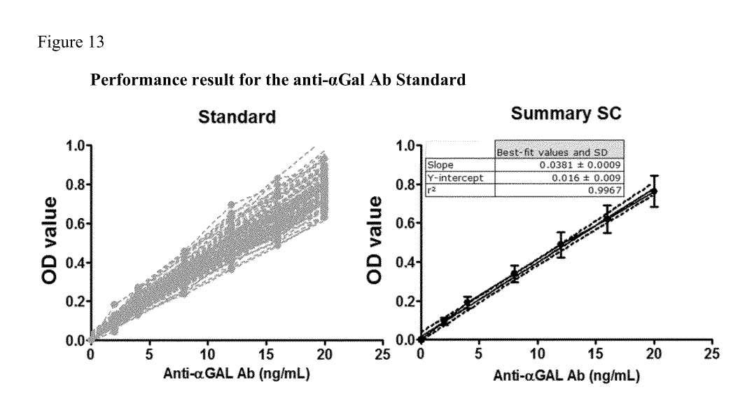

Each experiment conducted utilized the standard to determine the anti-αGal antibody values present in patient samples. FIG. 13 shows the values obtained for all the experiments and the summary for the performance of the anti-αGal antibody standard. As shown in FIG. 13, the variability and the coefficient of determination are within expected and acceptable range.

### Conclusion

The anti-αGal antibody ELISA for the testing of patient's samples was conducted according to guidelines published by the US Food and Drug Administration (FDA), 2001 and the ICH Harmonized Tripartite Guidelines, 2005. Results indicate that the performance of all quality controls utilized in this study have acceptable range of variability intra and inter assay. This study showed that the assay has high level of consistency, consequently it is considered adequate to support conclusions presented in this report.

### Example 4

### Detection of Anti-CEA Antibodies in Patients Enrolled in NLG0205 and NLG0305 Clinical Trials Introduction

CEA (Carcinoembryonic antigen) is a well-studied member of the immunoglobulin superfamily. CEA is a complex, highly glycosylated macromolecule containing approximately 50% carbohydrate, with a molecular weight of approximately 200 kDa. CEA was first discovered in human colon cancer tissue extracts. It is a useful marker for monitoring colon cancer after surgery and for monitoring treatment progression of lung and pancreatic cancer patients.

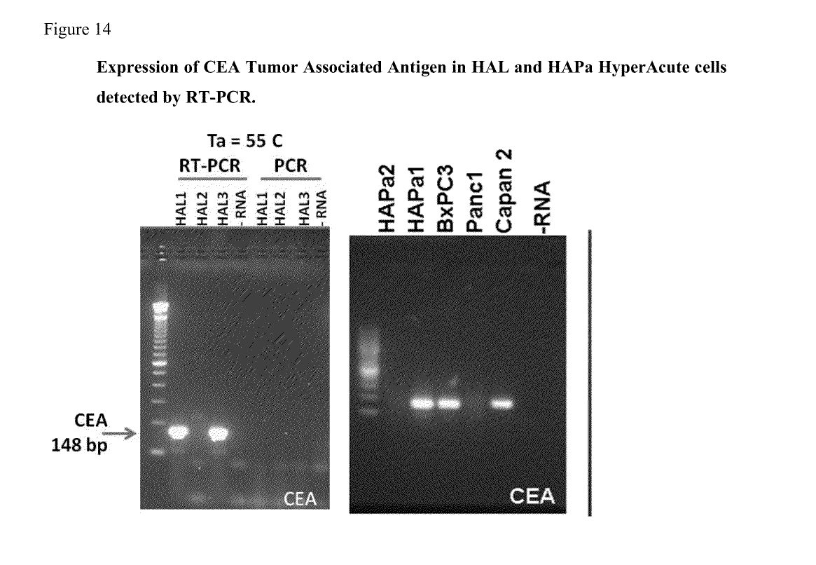

The HyperAcute immunotherapy is manufactured using cell lines genetically engineered to express αGal epitopes. Several of the cell line tested that are components of HyperAcute-Pancreas and Lung vaccines expresses high levels of CEA tumor antigen (FIG. 14).

We tested the development of anti-CEA antibodies in patients receiving HAPa immunotherapy before and after immunization as a mean of performing the immunological monitoring of evaluable patients and to determine if this biomarker could be correlated with better outcome.

### Brief Assay Description

The detection of anti-CEA antibodies was performed by ELISA. Briefly A 96-well microliter plate is coated with commercially available CEA antigen overnight, washed and blocked with buffer at 37° C. Samples (Primary Antibody) are dispensed on the plate, allowed to react with antigen and washed. Enzyme conjugated secondary antibody is dispensed on the plate and allowed to react with primary antibody. A chromogenic detection substrate is dispensed on plate and allowed to react with conjugate yielding a product with blue color. Reaction is stopped with 2M Sulphuric acid and optical density (OD) of samples is detected with a plate reader at a wavelength of 450 nm. Analysis of data is performed using Microsoft Excel and/or GraphPad Prism software. In each plate a qualified normal pooled serum sample (NPS10) is also tested as quality control reagent. All patients' samples are tested in each plate.

We tested the immunological response to CEA of patients enrolled in NLG0205. For analysis of the anti-CEA antibody, we selected samples collected before immunization, a sample after patients received 3 vaccinations, and a sample after they received all immunizations. Samples from the same patient were analyzed at the same time. All patients with available samples were evaluated.

We calculated the percent of change compared to baseline values according to the formula: Formula=Percent of change compare to baseline values (initial):

$$\frac{{N}_{final} - {N}_{initial}}{{N}_{initial}} ( 100 ) = \% change$$

### Results

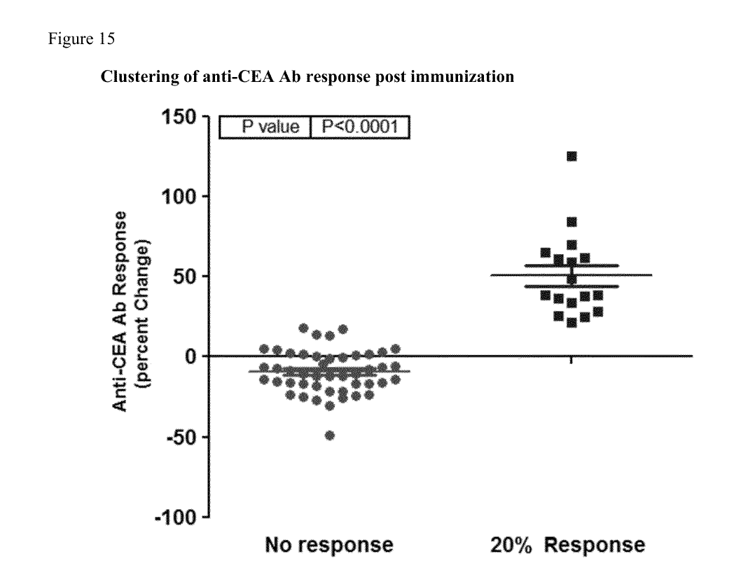

In this study we analyzed 63 patients with available samples before and after immunization. We observed a clustering of response that was characterized by a threshold of 20% increase in the response after immunization compared to baseline. This clustering of response was statistically significant and potentially clinically meaningful (p<0.0001) FIG. 15 shows the statistically significant clustering of the response post-immunization of evaluated patients.

In this study, 17 out of 63 patients showed a statistically significant increase in the anti-CEA antibody values post immunization. Table 5 below shows the summary of the response.

TABLE 5
Anti-CEA antibody response in patients receiving
Algenpantucel-L immunotherapy

| Anti-CEA Ab | | | |
|---|---|---|---|
| response | No Increased | Increase | Total |
| Counts | 46 | 17 | 63 |
| percent | 73 | 27 | |
| OS (Months) | 20.5 | 39.5 | |
| Survival Rate (%) | 33% | 41% | |

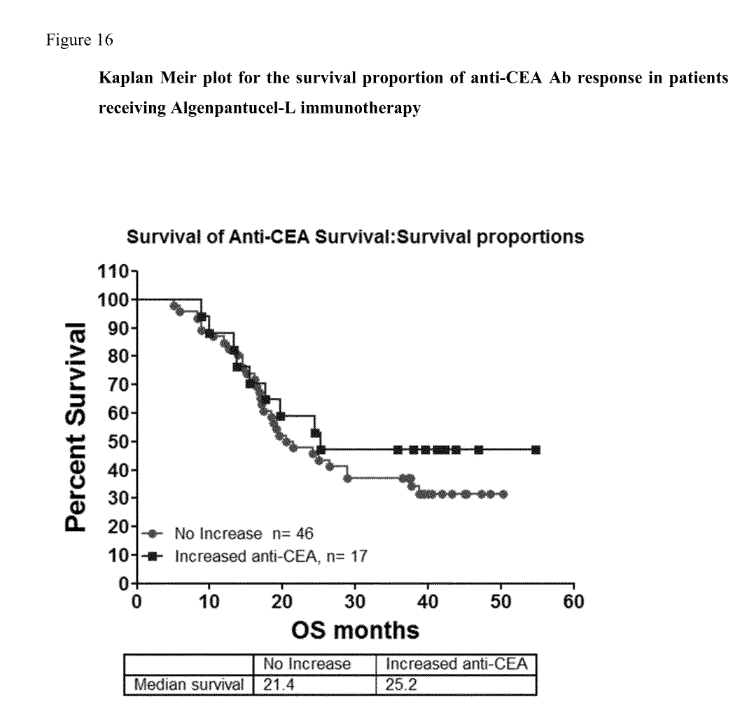

Patients that seroconverted to higher levels of anti-CEA antibodies after immunizations showed improved overall survival compared to patients with no increase in the anti-CEA antibody response (Table 5 and FIG. 16). FIG. 16 shows the survival proportions in a Kaplan-Meir plot of patients with and without increased anti-CEA antibodies after immunization.

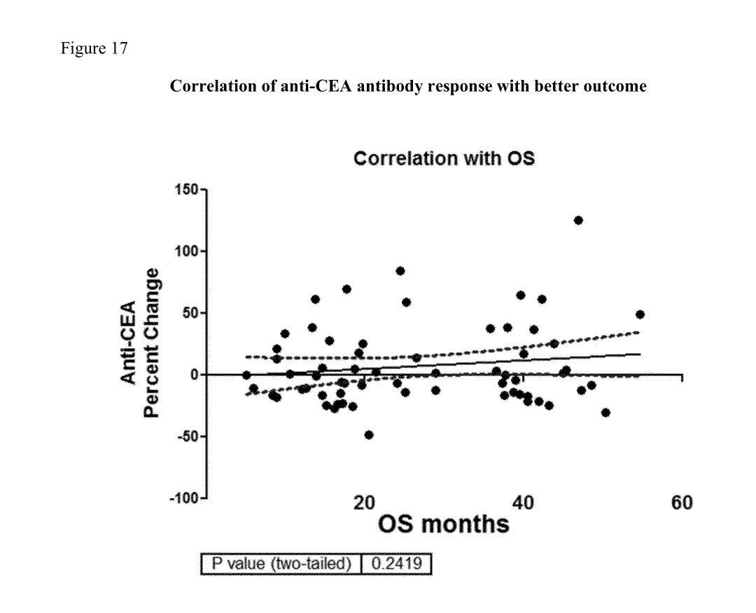

To determine if the magnitude of the response anti-CEA had a correlation with better outcome we performed a Pearson correlation calculation. FIG. 17 demonstrates that there is not a statistically significant correlation between the development of higher levels of anti-CEA antibody and better outcome indicating that the response (sero-conversion) and not the magnitude of the response is associated with better outcome.

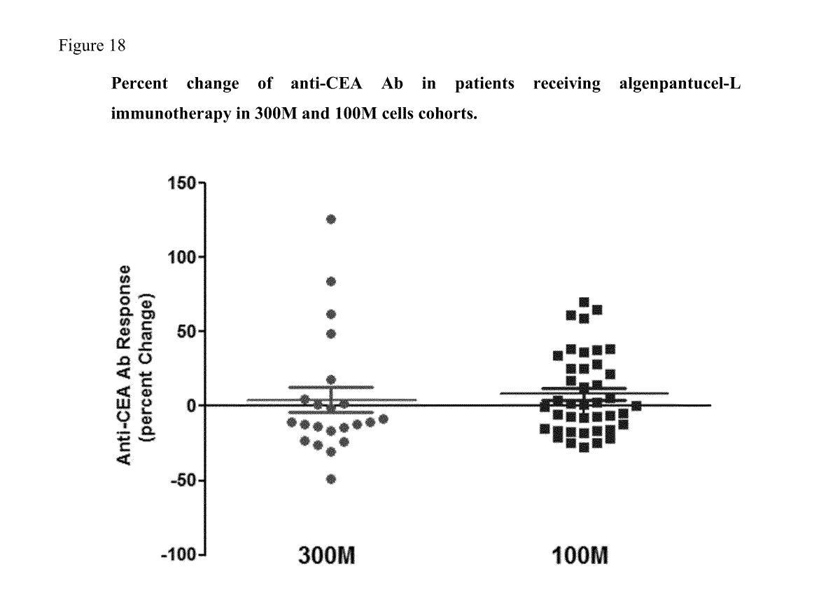

We compared the percentage of change in the anti-CEA antibody levels in patients receiving 300M cell vaccines or 100M cell vaccine (FIG. 18). As shown in FIG. 18, there is no difference in the type of response observed in patients receiving either dose suggesting that at least concerning the anti-tumor immune response measured by the change in the levels of this antibody, both dose regimes seem similar.

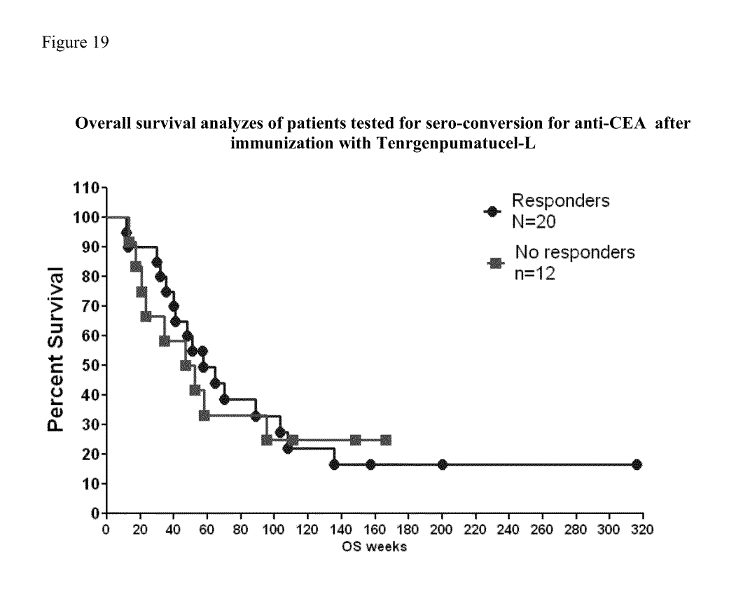

In a separate trial testing the efficacy Tenrgenpumatucel-L (HyperAcute® Lung Immunotherapy) in lung cancer, 32 patients have been tested for the presence of anti-CEA antibodies before and after immunization. The patients analyzed have received more than two immunizations. Of the 32 patients analyzed, 20 patients responded with significant increased anti-CEA antibody values after immunization, although three did exhibit a slight decrease in anti-CEA antibodies during immunization (considered not changed in the analysis). Preliminary analysis of the overall survival of patients analyzed in this study suggest no predictive or favorable correlation in patients responding with increased anti-CEA antibody titer compared to patients with no change in the anti-CEA antibody levels (FIG. 19).

### Anti-CEA Antibody ELISA Assay Performance

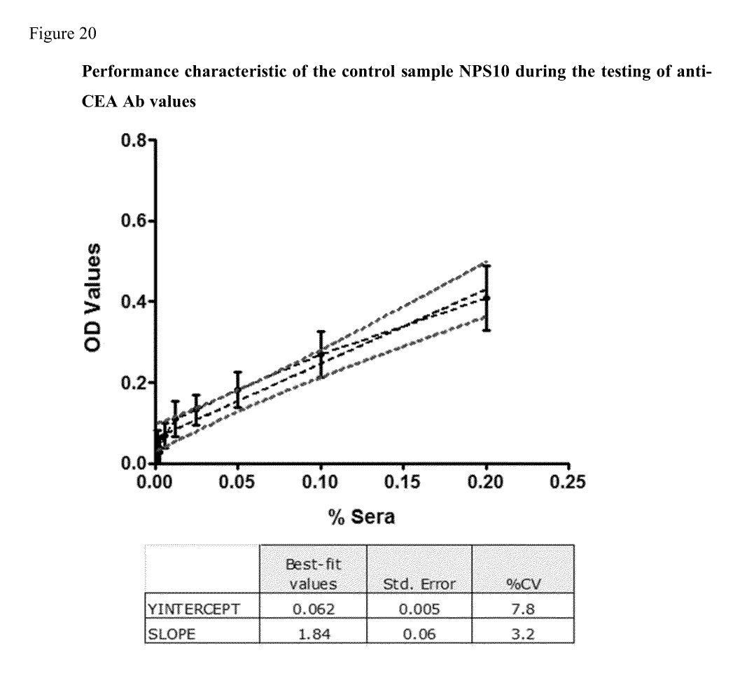

To monitor the quality of the assay, a qualified normal pool sera sample (NPS10) was tested in each experiment in each plate. Data below shows the performance of the NPS10 control sample during the course of this study (FIG. 20). As shown in FIG. 20, testing of NPS10 was consistent with less than 10% CV for the determination of both the slope and the y-intercept, demonstrating that the values obtained during this study have acceptable quality with variation among experiments within acceptable range.

### Conclusions

Production of anti-CEA antibodies was elevated in 17 out of 63 (27%) patients evaluated after immunotherapy. The elevation of anti-CEA antibody was associated with better outcome. Patients responding with anti-CEA antibody had median overall survival (OS) of 25.2 months comparing favorably to patients without sero-conversion which had a median OS of only 21.4 months. In addition, it is feasible to perform the immunological monitoring of Algenpantucel-L immunotherapy trials detecting anti-CEA antibody and possibly use this biomarker as surrogate marker to determine efficacy in Algenpantucel-L clinical trials. Furthermore, in trials of other cancers treated with different HyperAcute immunotherapy, the presence of increased titers of anti-CEA antibodies does not correlate with an improved overall survival (data not shown).

### Example 5

### Detection of Anti-Mesothelin Antibody in Patients Enrolled in NLG0205 Introduction

Most tumor-associated antigens are expressed in greater extent in cancer tissues as compared to normal tissues. This may help the immune system to recognize the over-expressed genes in tumor. Hence, over-expressed genes have potential to be immunogenic and can be targeted for immunotherapy.

Mesothelin is a 40 kDa differential antigen which is expressed on normal mesothelial cells and over-expressed in various cancer including pancreatic, cervix, esophagus, lung, ovarian cancers and mesotheliomas (Chang et al., PNAS, 1996; Ordonez et al., Mod Path. 2003; Ordonez et al. Am J Surg Pathol, 2003; Argani et al. 2001; Ho et al. 2007). Its expression is tested with SAGE (Argani et al. 2001) and immunohistochemistry (IHC) using monoclonal antibody K1 (Chang et al., Int J Cancer, 1992; Chang et al., Am J Surg Pathol, 1992) and later commercial antibody 5B2 (Ordonez et al., Mod Pathol, 2003; Ordonez et al, Am J Surg Pathol, 2003). The precursor of mesothelin is a 69 kDa protein that is processed into 40 kDa membrane-bound mesothelin and 31 kDa shed protein known as megakaryocyte-potentiating factor that is secreted from the cells and identified from the medium of human pancreatic cancer cell line (Yamaguchi et al., J Biol Chem, 1994).

The biological function of mesothelin is not clear. Deletion of both copies of mesothelin had no abnormalities in the mutant mice as compared to wild-type mice (Bera et al, Mol Cell Biol, 2000). Mesothelin has been suggested to play a role in adhesion because 3T3 cells transfected with mesothelin were more adherent to the culture dishes than non-transfected cells (Chang et al, PNAS, 1996). It is supported by the study showing mesothelin interaction with CA125 which might play role in metastasis of tumor (Rump et al, J. Biol. Chem, 2004; Gubbels et al., Mol Cancer, 2006).

Its limited expression in normal cells makes it an attractive target for immunotherapy (Hassan et al. Clin Cancer Res, 2004) Mesothelin antibodies are present in the sera of patients with mesothelin expressing cancers (Ho et al., Clin Cancer Res, 2005). It has also shown to elicit T-cell response (Yokokawa et al, Clin Cancer Res, 2005; Thomas et al, J Exp Med, 2004). There are various clinical trials going on that target mesothelin or elicit immune response against mesothelin. A recombinant immunotoxin (SS1P) containing an anti-mesothelin Fv linked linked to truncated exotoxin has shown to mediate cell killing of mesothelin-expressing cells and tumors (Ho et al, Clin Cancer Res, 2007; Hassan et al, Clin Cancer Res, 2004; Hassan et al., Clin Cancer Res, 2006). Two Phase I clinical trials have been recently completely for SS1P (Hassan et al. Clin Cancer Res, 2007). Another clinical trial by Jaffe et al involved vaccinating pancreatic cancer patients with GM-CSF transduced pancreatic cancer cell lines (Jaffee et al., J Clin Oncol, 2001). 3 out of 14 patients developed post-vaccination delayed type-hypersensitivity associated with prolonged survival. Following immunological studies showed that these 3 patients strong induction of CD8+ T cell response (Thomas et al., J Exp Med, 2004). These studies support mesothelin as a promising target for immunotherapy.

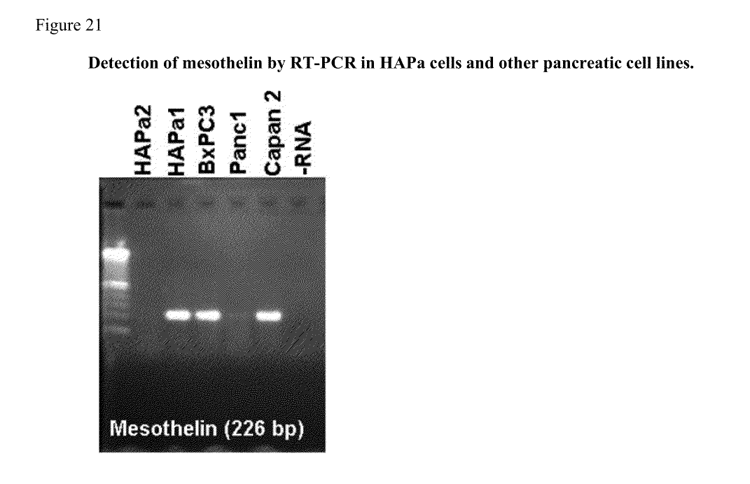

Algenpantucel-L immunotherapy is manufactured using two pancreatic cell lines genetically engineered to express αGal epitopes. One of the cell line components of Algenpantucel-L immunotherapy, HAPa1, expresses high levels of mesothelin antigen by RT-PCR (FIG. 21).

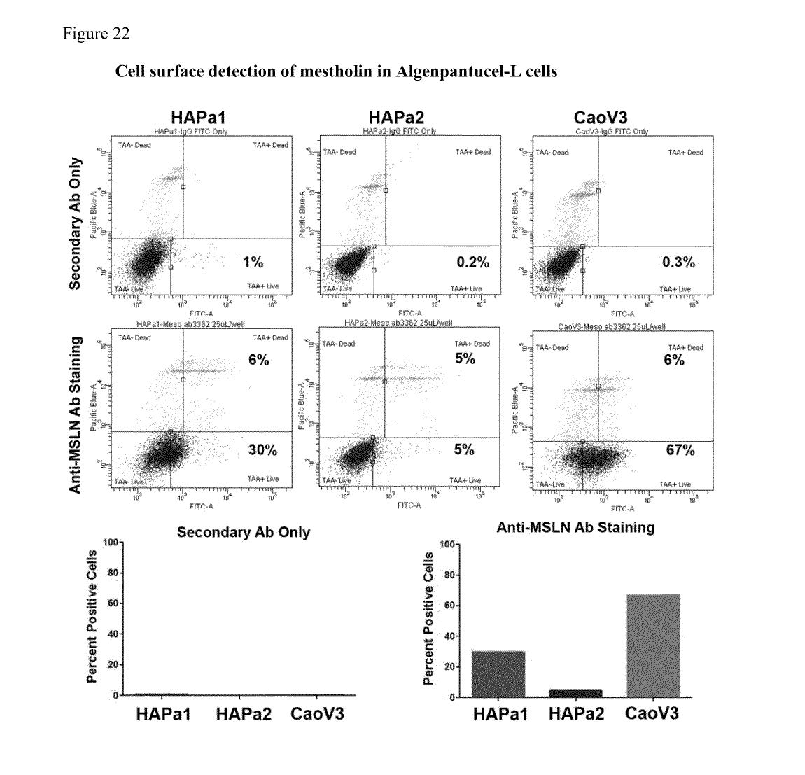

In addition, membrane bound mesothelin can be detected by FACS analysis in HAPa1 cells. FIG. 22 shows the staining of Algenpantucel-L cells. As a positive control we stained an ovarian cancer cell line (CaoV3) that shows high expression of mesothelin.

### Brief Assay Description

The detection of anti-Mesothelin (MSLN) antibodies was performed by ELISA. Briefly A 96-well microliter plate is coated with recombinant purified membrane bound mesothelin (antigen) overnight, washed and blocked with buffer at 37° C. Samples (Primary Antibody) are dispensed on the plate, allowed to react with antigen and washed. Enzyme conjugated secondary antibody is dispensed on the plate and allowed to react with primary antibody. A chromogenic detection substrate is dispensed on plate and allowed to react with conjugate yielding a product with blue color. Reaction is stopped with 2M Sulphuric acid and optical density (OD) of samples is detected with a plate reader at a wavelength of 450 nm. Analysis of data is performed using Mircrosoft Excel and/or GraphPad Prism softwares. In each plate a qualified normal pooled serum sample (NPS10) is also tested as quality control reagent. All patients samples are tested in each plate.

We tested the immunological response to membrane bound mesothelin of patients enrolled in NLG0205. For analysis of the anti-MSLN antibody, we selected samples collected before immunization, a sample after patients received 3 vaccinations, and a sample after they received all immunizations. Samples from the same patient were analyzed at the same time. All patients with available samples were evaluated.

We calculated the percent of change compared to baseline values according to the formula:
Formula=Percent of change compare to baseline values (initial):

$$\frac{{N}_{final} - {N}_{initial}}{{N}_{initial}} \cdot ( 100 ) = \% change$$

### Results

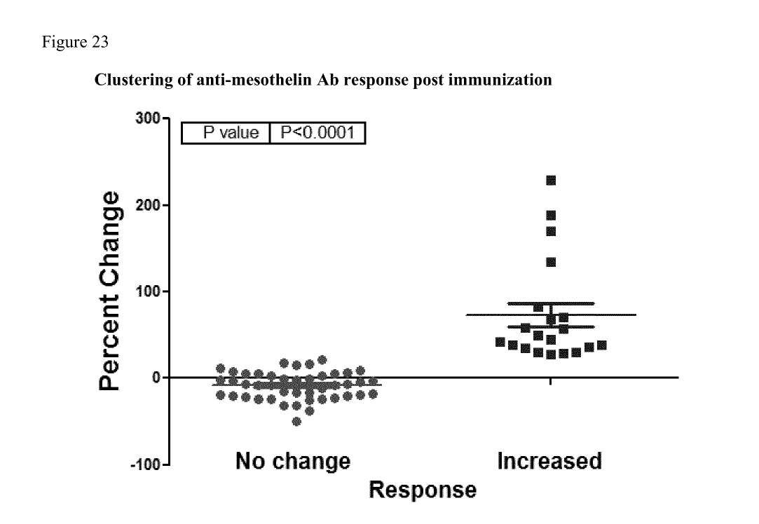

In this study we analyzed 64 patients with available samples before and after immunization. We observed a clustering of anti-mesothelin antibody response that was characterized by a threshold of 25% increase in the response after immunization compared to baseline. FIG. 23 shows the statistically significant clustering of the response post-immunization of evaluated patients.

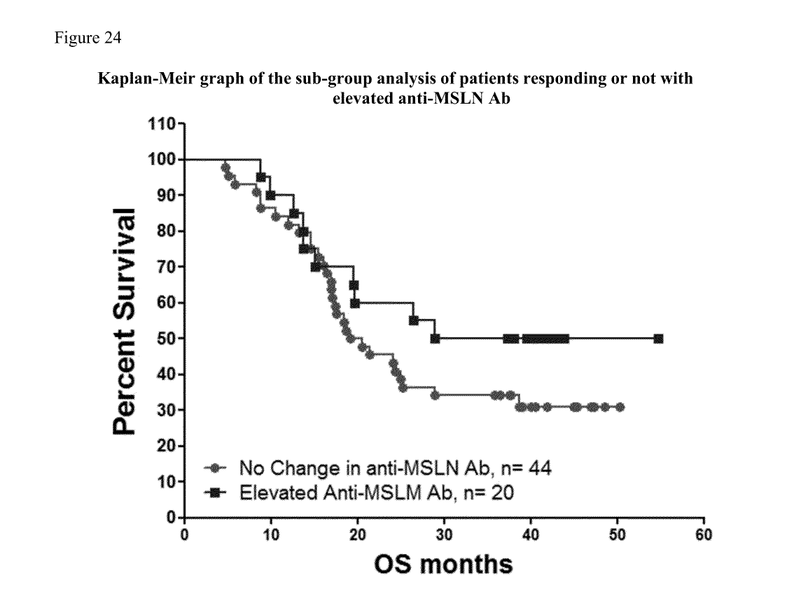

Of the 64 patients evaluated, 20 (31%) patients had an increased anti-MSLN antibody response after immunization. Patients that seroconverted to anti-MSLN antibody had a better outcome with a median overall survival of 42 months compared to patients that had no anti-MSLN antibody response after immunization which had a median overall survival of 20 months (Table 6, and FIG. 24).

TABLE 6
Anti-Mesothelin (MSLN) antibody
response of evaluated patients

| Anti-MSLN Ab | No Increase | Increase | total |
|---|---|---|---|
| counts | 44 | 20 | 64 |
| percent | 69 | 31 | |
| OS (Months) | 20 | 42 | |
| Survival Rate | 31% | 50% | |

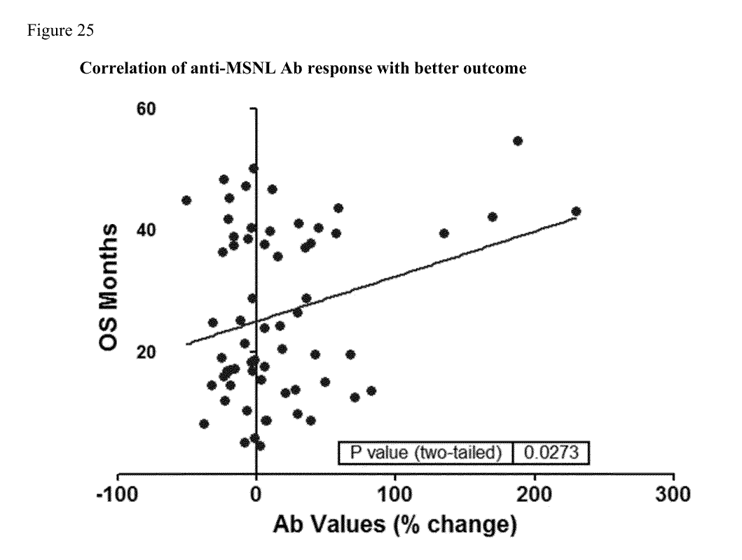

To determine if the magnitude of the response anti-mesothelin antibody had a correlation with better outcome we performed a Pearson correlation calculation. FIG. 25 demonstrated that there is a statistically significant correlation between the development of anti-MSLN antibody and better outcome.

### Anti-MSNL Antibody ELISA Assay Performance

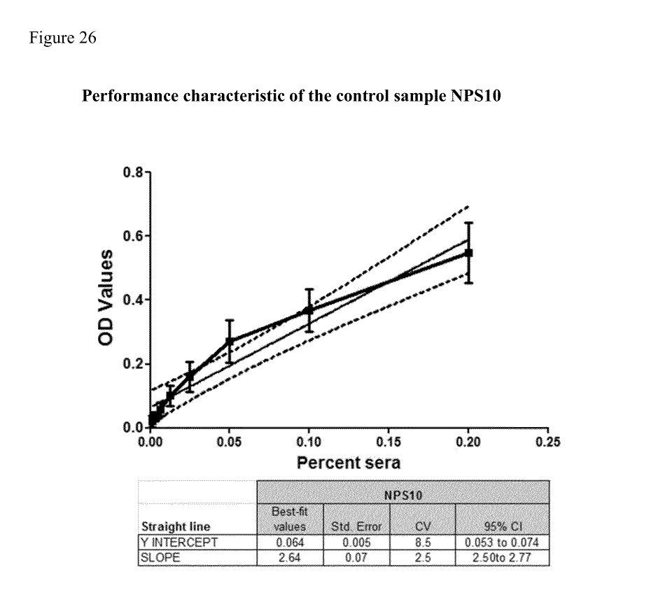

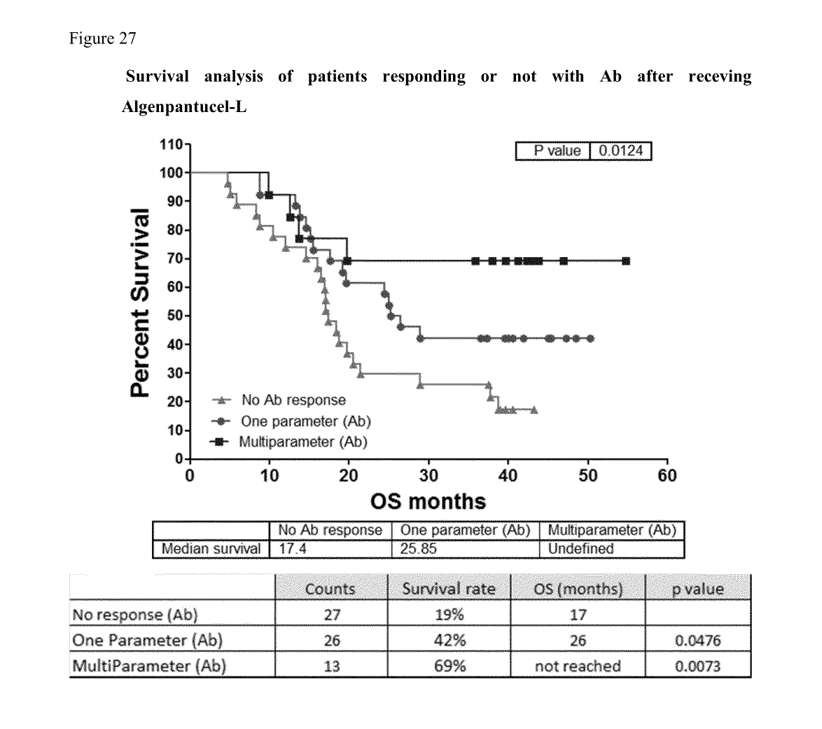

To monitor the quality of the assay, a qualified normal pool sera sample (NPS10) was tested in each experiment in each plate. FIG. 26 shows the performance of the NPS10 control sample during the course of this study. As shown in FIG. 27, testing of NPS10 was proven to be consistent with less than 10% CV for the determination of both the slope and the y-intercept, demonstrating that the values obtained during this study have acceptable quality with variation among experiments within acceptable range.

### Conclusions

Production of anti-MSLN antibodies was elevated in 20/64 (31%) patients evaluated after immunotherapy. The elevation of anti-MSLN antibodies was correlated with better outcome (p<0.03). Patients responding with anti-MSLN antibodies had median overall survival of 42 months comparing favorably to patients without sero-conversion which had a median overall survival of only 20 months. In addition, it is feasible to perform the immunological monitoring of Algenpantucel-L immunotherapy trials detecting anti-mesothelin antibodies and possibly use this biomarker as surrogate marker to determine efficacy in Algenpantucel-L clinical trials.

### Example 6

### Multivariate Analysis of the Humoral Immune Response in Patients Receiving Algenpantucel-L Immunotherapy

We performed a preliminary exploratory data analysis combining observations accrued during this study to determine if the development of either antibody alone or in combination might have a predictive value on treatment progression and or possibly early signs of response. Results from this analysis might have significant value to determine at early time points during the study if the immunotherapy has a higher or lower probability of success. The knowledge at early time points of probability might have an impact on subsequent treatment decisions.

In this analysis patients were categorized in three groups:
1. Patients which showed no significant response to antibody production. These patients have less than 10 fold-increase in the anti-αGal antibody, they have less than 20% increase in anti-CEA antibody production and less than 25% increased anti-mesothelin antibody production after immunization,
2. Patients with response to one type of antibody studied. They responded either with more than 10 fold-increase in the anti-αGal antibody values or anti-CEA (>25%) or anti-Mesothelin antibody (>25%).
3. Patients that responded with two or more types of antibody production. These patients have a combination of either two parameters studied or they have increased in the three types of antibodies studied. The matrix of possible combinations for this group of patients is shown in Table 7:

TABLE 7
matrix of possible combinations for patients
responding to multiple parameters

| | aGAL | CEA | MSNL | CEA and MSLN |
|---|---|---|---|---|
| aGAL | | x | x | x |
| CEA | | | x | |
| MSLN | | | | |

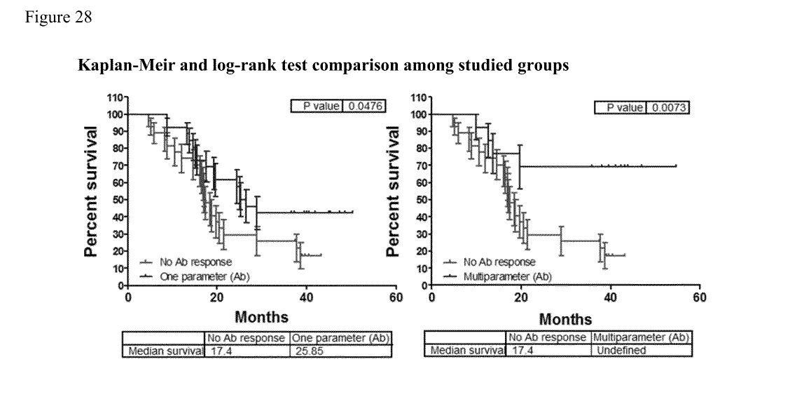

Sixty six patients were evaluated in this multivariate analysis. Results demonstrate that a statistically significant favorable median overall survival is observed in patients responding with antibody after immunization (p=0.012). Patients responding with one type of antibody studied (n=26) had a significantly better outcome compared to patients with no antibody response (n=27) after immunization (26 months vs 17 months, p=0.047). Moreover patients responding with two or more types of antibodies had an even better outcome. In this group of patients the median survival has not been reached yet (as of 01-23-2013). FIG. 28 shows the Kaplan-Meir plot for the three groups described. Importantly the majority of patients who responded with increased antibody titers after immunization early during treatment in general did so after the first two immunizations. Consequently these data has the potential to predict the course therapy early during the administration of the immunotherapy FIG. 28 shows the comparison of median survival including the confidence intervals of groups. Table 8 summarizes the findings.

TABLE 8
Summary of the multivariate analysis for
Algenpantucel-L immunotherapy trial

| | Counts | Survival rate | OS (months) | p value |
|---|---|---|---|---|
| No response (Ab) | 27 | 19% | 17 | |
| One Parameter (Ab) | 26 | 42% | 26 | 0.0476 |
| MultiParameter (Ab) | 13 | 69% | not reached | 0.0073 |

### Conclusions

In this multivariate analysis we determined that the likelihood of responding to therapy is significantly greater if response to antibodies is observed. The antibody response is observed early during the course of immunization, consequently this data potentially could be use a method to change the course of treatment or might help in the assistance of decisions for subsequent therapies.

### Example 7

### Correlation of Increased Eosinophil Levels With Overall Survival After Immunization With Algenpantucel-L

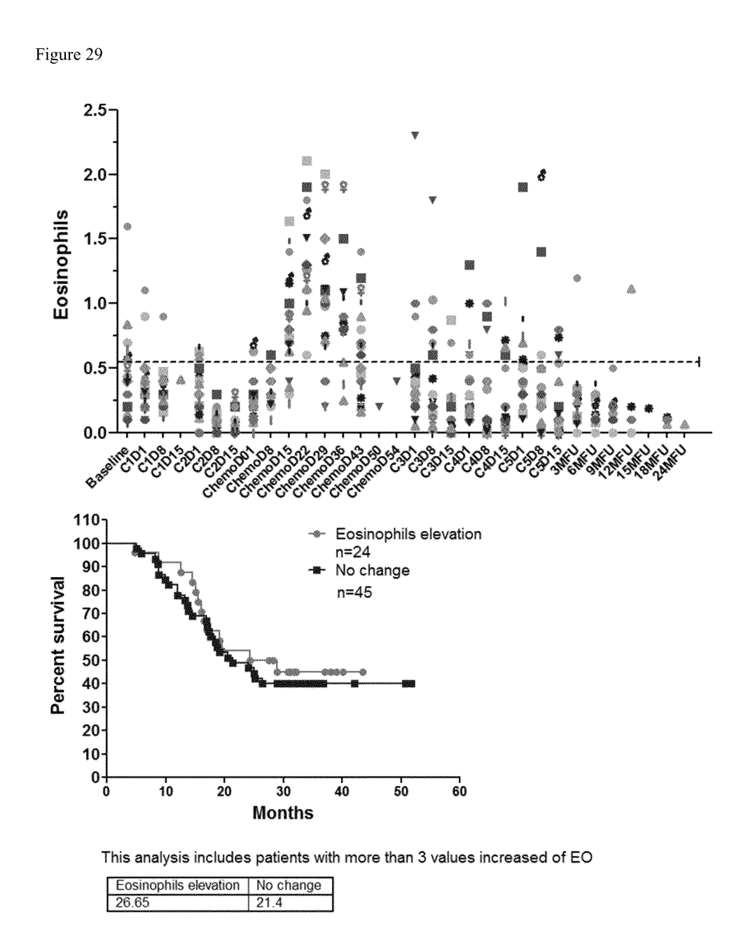

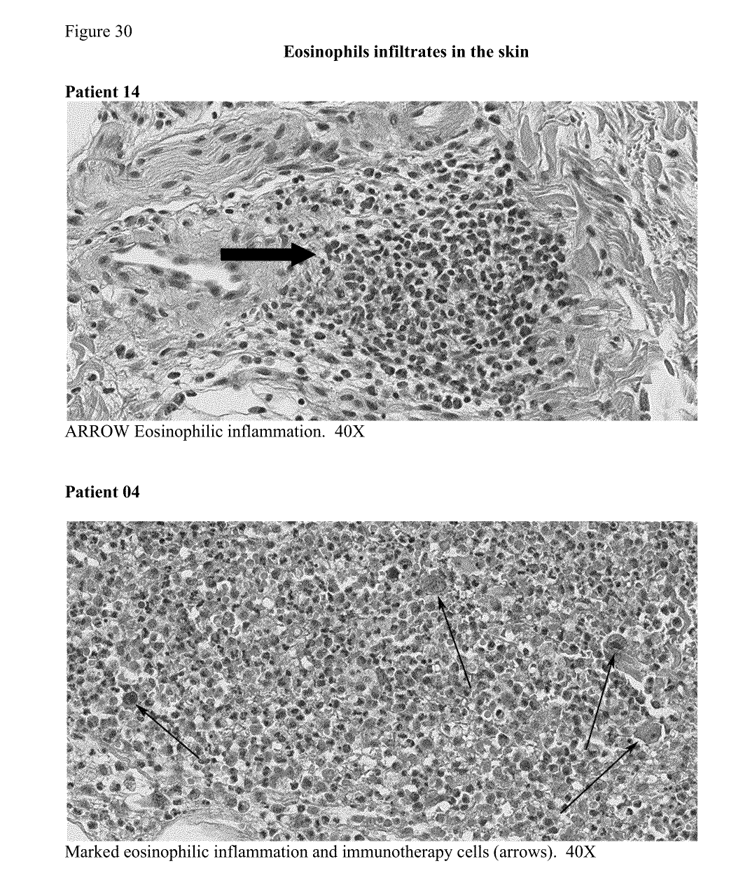

After administration of pancreatic cancer patients with Algenpantucel-L some patients demonstrated an increase in eosinophils that correlates with an improved patient outcome. Patients that exhibited this increase in eosinophil levels at least three times during the course of immunization have a median survival of 27 months compared to 21 months for patients with no elevation of eosinophils (FIG. 29). In addition, there is evidence that skin inflammatory reactions surrounding the skin biopsies at the injection site show eosinophil infiltrates. The presence of eosinophils at the injection sites might be unique to Algenpantucel-L (FIG. 30).

### Example 8

### Correlation of Increased Production of Anti-Calreticulin Antibodies With Overall Survival After Immunization With Algenpantucel-L

Calreticulin (CALR) is a multifunctional protein located in storage compartments associated with the endoplasmic reticulum. Calreticulin binds to misfolded proteins and prevents them from being exported from the endoplasmic reticulum to the Golgi apparatus. A similar quality-control chaperone, calnexin, performs the same service for soluble proteins as does Calreticulin (Ellgaard et al. 2003). CALR is expressed on the cell surface of many cancer cells and plays a role to promote macrophages to engulf cancerous cells (Chaput et al. 2007; Obeid et al. 2007). When CALR is exposed on the cell surface, it also serves as a signal that allows a dying cell to be recognized, ingested and processed by specialized phagocytic and dendritic cells, which educate other immune cells to recognize and respond to the material they have ingested generating an immune response (Obeid et al. Nature Medicine, 2007). CD47, which blocks CALR prevents destruction of most of the cells. Phagocytic cells recognize CALR by LDL receptor-related protein (LRP-1- or CD91).

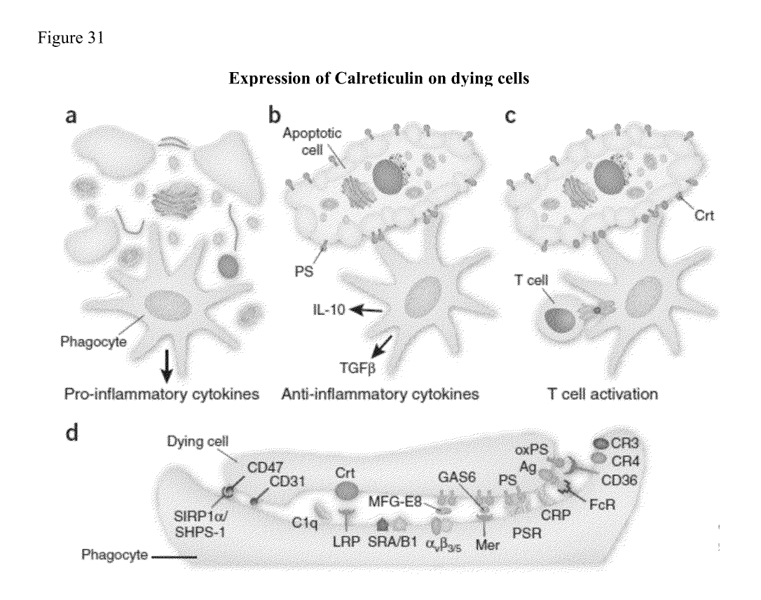

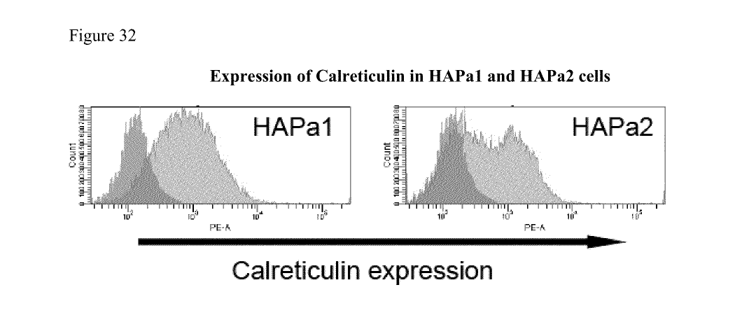

Dying cells play an important role in the generation of an immune response. FIG. 31 shows different receptors present on dying cells. In lytic/necrotic death (A) fragments of cells that die by necrosis are taken up by phagocytes, which can trigger production of pro-inflammatory cytokines, leading to immune activation and, potentially, autoimmunity. Apoptotic cells (B) are recognized by cell surface markers such as phosphatidyl serine (PS) and phagocytosed and undergo non-immunogenic death which can cause phagocytes to release anti-inflammatory molecules (e.g., IL-10 and TGFβ). However, apoptotic cells that display calreticulin on their surface are processed by dendritic cells that induce a specific T cell-mediated immune response against these apoptotic cells (C). Phagocytosis of apoptotic cells is determined by a combination of cell surface markers. Viable cells display inhibitory signals (including CD47, which interacts with SHPS-1 on the phagocyte, and CD31) that disappear from the cell surface upon apoptosis induction. During apoptosis, cells expose markers that stimulate phagocytosis, including phosphatidyl serine (PS), recognized by phosphatidyl serine receptors (PSR); calreticulin (CALR), recognized by low-density lipoprotein-receptor-related protein (LRP) and C1q; and oxidized phosphatidyl serine (oxPS), recognized by CD36 (D. FIG. 31). FIG. 32 shows that calreticulin is expressed on both HAPa1 and HAPa2 cells.

Based on this information we postulated that the expression of Calreticulin in Algenpantucel-L drug product will induce an immunological reaction to Calreticulin that could be detected after immunization. These “de-novo” antibodies might be potentially used as surrogate markers for vaccine efficacy.

The purpose of this study was to determine the reactivity anti-calreticulin detected in patients receiving Algenpantucel-L in NLG0205 clinical study. In addition the development of anti-calreticulin antibodies was correlated with clinical outcome to determine if the development of anti-calreticulin antibodies has a potential predictive value for clinical efficacy.

The detection of anti-Calreticulin (CALR) antibodies was performed by ELISA. A 96-well microliter plate is coated with 50 μL/well of a 5 ug/mL CALR (Calreticulin Protein Fitzgerald Cat#80R-1306) overnight, washed and blocked with buffer at 37° C. Samples (Primary antibodies) were dispensed on the plate, allowed to react with antigen and washed. Enzyme conjugated secondary antibody was dispensed on the plate and allowed to react with the primary antibodies. A chromogenic detection substrate was dispensed on plate and allowed to react with conjugate yielding a product with blue color. The reaction was stopped with 2M Sulphuric acid and optical density (OD) of samples was detected with a plate reader at a wavelength of 450 nm. Analysis of data was performed using Mircrosoft Excel and/or GraphPad Prism softwares. In each plate a qualified normal pooled serum sample (NPS10) was also tested as quality control reagent.

The immunological response to CALR of patients enrolled in NLG0205 was studied. Serum samples were collected on day 1 of cycle #1 (before immunization-baseline), day 1 of cycle #2 (S2), days 1 (S3) and 43 (S4) of chemoradiation, day 1 of cycle #3 (S5), day 1 of cycle #4 (S6), day 1 of cycle #5 (S6), and at every follow-up visit (S7 and so on). Three samples were tested to analyze the anti-CALR antibodies produced: S1 (baseline), S3 (patients received 4 immunizations) and S6 (patients received about 12 immunizations).

FIG. 1 shows the protocol schedule. Samples from the same patient were analyzed at the same time. Samples from a patient were analyzed in a single plate. All patients with available samples were evaluated. The percent of change compared to baseline values was calculated according to the following formula using OD values in the lineal portion of the curve using serial dilutions.

$$\frac{{N}_{final} - {N}_{initial} ( 100 )}{{N}_{initial}} = \% change .$$

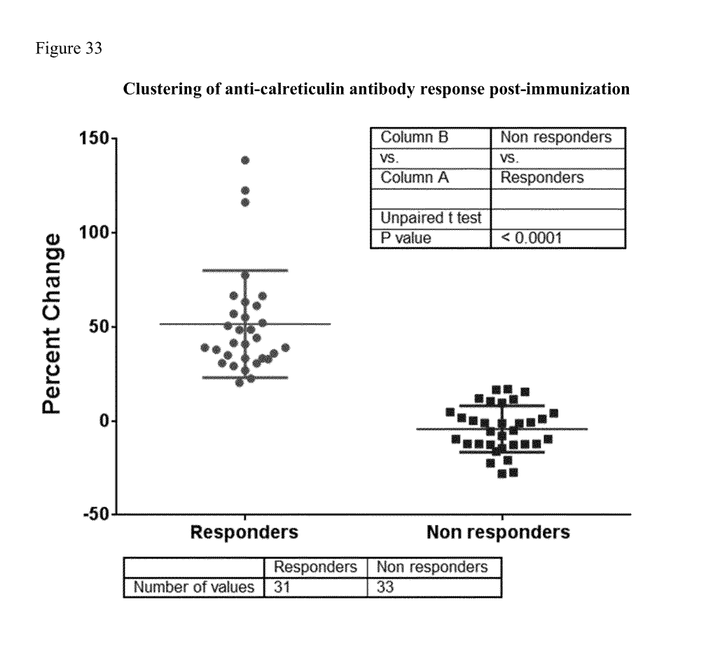

In this study 64 patients with available samples before and after immunization were analyzed. A clustering of response that was characterized by a threshold of 20% increase in the response after immunization compared to baseline was observed. FIG. 33 shows the statistically significant clustering of the response post-immunization of evaluated patients. Of the 64 patients evaluated, 31 (48%) patients had increased anti-CALR antibody response after immunization.

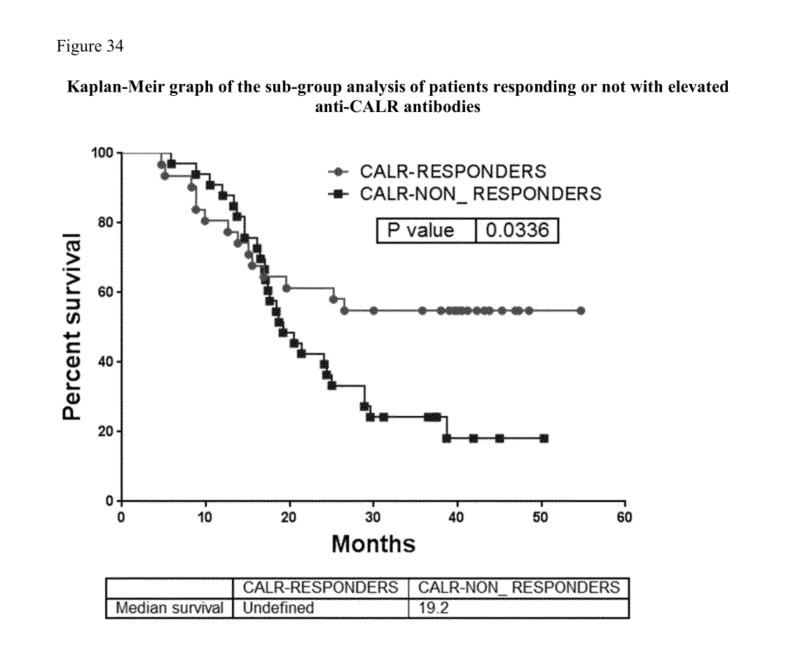

The median overall survival of patients responding with anti-CALR antibodies after immunization compared to the median overall survival of patients without sero-conversion was also evaluated. Patients that had increased anti-CALR antibodies after immunization had a significantly better outcome with a median overall survival over 35 months compared to patients that had no meaningful increased in the anti-CLR antibodies response after immunization (median overall survival 19.2 months FIG. 34). The difference in median overall survival was statistically significant (p<0.04).

Table 9 shows the survival rate analysis for patients responding with anti-CALR antibodies compared to patients with no meaningful increase in the anti-CALR antibody response. The statistical analysis indicates a significant difference in the outcome of patients responding with anti-CALR antibodies (Fisher exact test, p<0.01).

TABLE 9
Survival rate and fisher exact
test for anti-CALR Ab response

| Anti-CALR Ab | Increase Ab | No Increase | Total |
|---|---|---|---|
| Counts | 31 | 33 | 64 |
| OS (months) | >35 | 19.2 | p < 0.04 (log rank test) |
| Survival rate | 55% | 21% | p < 0.01 (Fisher's exact test) |

### Conclusions

Production of anti-CALR antibodies was elevated in 31/64 (48%) patients evaluated after immunotherapy. The elevation of anti-CALR antibodies was correlated with better outcome (p<0.04). Patients responding with anti-CALR antibodies had median overall survival (OS) of >35 months comparing favorably to patients without sero-conversion which had a median OS of only 19.2 months. In addition, it is feasible to perform the immunological monitoring of Algenpantucel-L immunotherapy trials detecting anti-CALR antibodies and possibly use this biomarker as surrogate marker to determine efficacy in algenpantucel-L clinical trials.

### Anti-CALR Ab ELISA Assay Performance

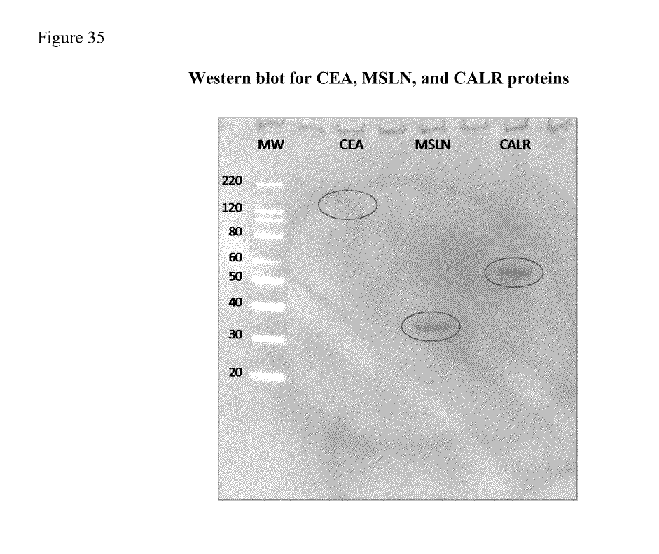

To monitor the quality of the assay and establish criteria for valid test, a qualified normal pool sera sample (NPS10) was tested in each experiment in each plate in parallel to patient's samples. This reagent was tested by western blot and showed specific reactivity against Calreticulin. FIG. 35 shows the reactivity of NPS10 anti-CALR, anti-CEA and anti-Mesothelin detected by western blot. Consequently this reagent is considered suitable to use as a control for this assay.

The performance of NPS10 was evaluated INTRA and INTER experiments during the testing of samples described before.

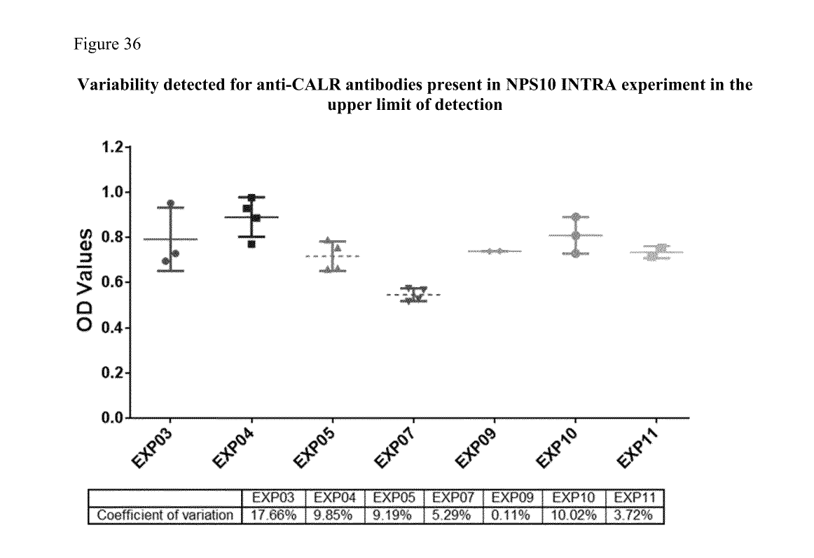

FIG. 36 shows the variability in the detection of NPS10 reactivity against Calreticulin intra experiment in the upper limit of detection of this assay. As shown in FIG. 36, the variability (coefficient of variation) for the upper limit of detection of anti-CALR antibodies present in NPS10 is below 10% in all experiments except EXP03, where the variability observed was 17.66%. The variability in the detection of the anti-CALR antibodies present in NPS10 inter experiment was evaluated during the course of this study.

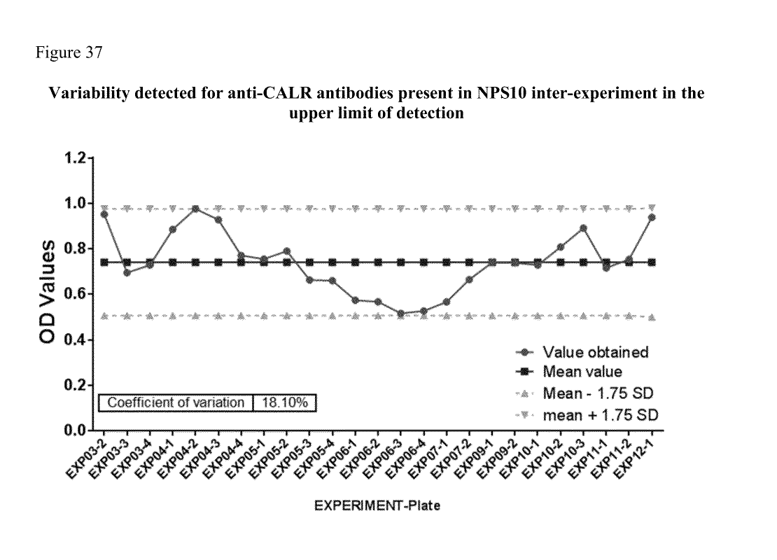

As shown in FIG. 37, the variability observed INTER experiment was 18%. FIG. 37 shows each individual value for each experiment. The box line represents the mean of all experiments. The triangle lines represent mean value expected plus or minus 1.75 SD. Experiments were considered valid for the detection of NPS10 anti-Calreticulin antibodies when the values observed were within this range.

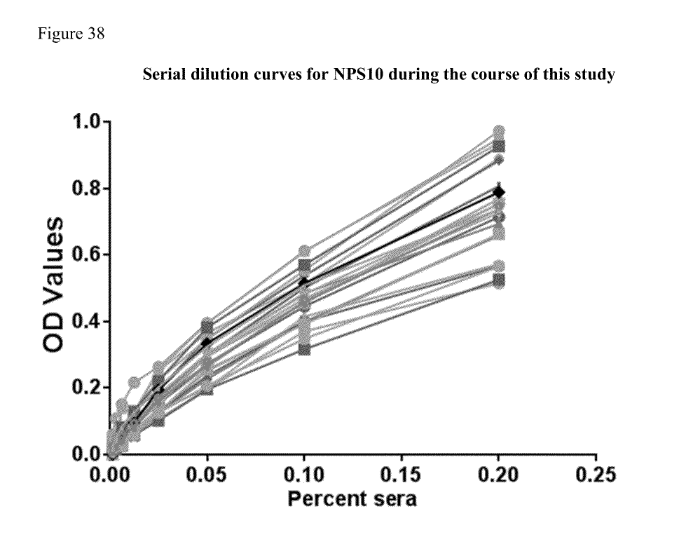

Each serum sample was subjected to serial dilutions in order to obtain OD values in a linear portion of the curve to perform the calculation of the percent change compared to baseline. Similarly, serial dilutions are performed for NPS10 as a quality control. FIG. 38 shows corresponding OD values for NPS10 in each plate and each experiment.

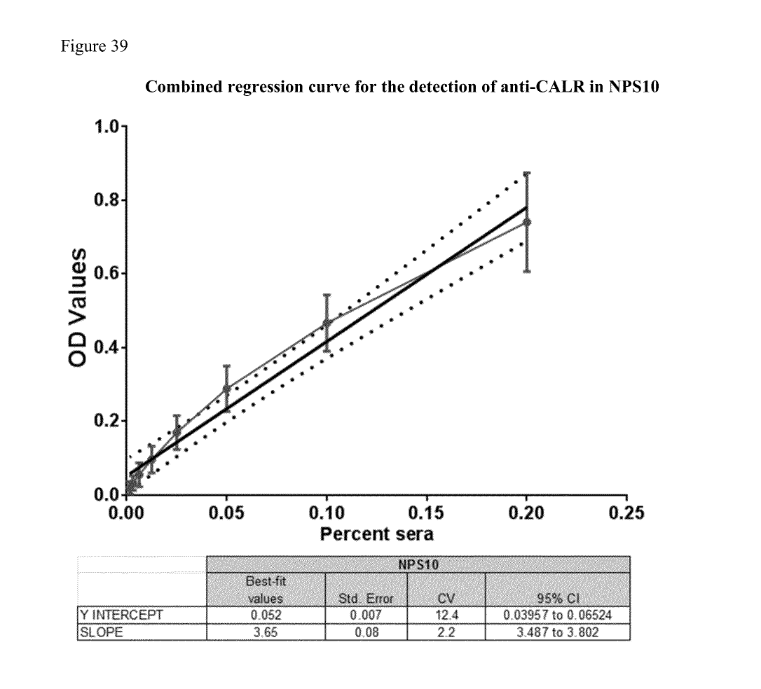

In order to evaluate the variability inter-experiment combined values were fitted to a linear regression (FIG. 39). FIG. 39 shows the average value for each point with error bars as SD. The fitted curve is also shown with the 95% CI. As demonstrated in FIG. 39, the combined values for the Y-intercept and slope have acceptable precision demonstrating reproducibility in the assay.

Testing of NPS10 for the presence of anti-CALR antibody reactivity was proven to have acceptable reproducibility with % CV intra-experiment below 10% and inter-experiment variability below 20%. Consequently the assay is considered suitable for detecting anti-CALR antibody reactivity in serum samples. While specific embodiments of the invention have been described and illustrated, such embodiments should be considered illustrative of the invention only and not as limiting the invention as construed in accordance with the accompanying claims.

All patents, applications, and other references cited herein are incorporated by reference in their entireties.

### REFERENCES

1. Chang, K. and I. Pastan, Molecular cloning of mesothelin, a differentiation antigen present on mesothelium, mesotheliomas, and ovarian cancers. Proc Natl Acad Sci USA, 1996. 93(1): p. 136-40.

2. Ordonez, N. G., Value of mesothelin immunostaining in the diagnosis of mesothelioma. Mod Pathol, 2003. 16(3): p. 192-7.

3. Ordonez, N. G., Application of mesothelin immunostaining in tumor diagnosis. Am J Surg Pathol, 2003. 27(11): p. 1418-28.

4. Argani, P., et al., Mesothelin is overexpressed in the vast majority of ductal adenocarcinomas of the pancreas: identification of a new pancreatic cancer marker by serial analysis of gene expression (SAGE). Clin Cancer Res, 2001. 7(12): p. 3862-8.

5. Ho, M., et al., Mesothelin expression in human lung cancer. Clin Cancer Res, 2007. 13(5): p. 1571-5.

6. Chang, K., I. Pastan, and M. C. Willingham, Isolation and characterization of a monoclonal antibody, K1, reactive with ovarian cancers and normal mesothelium. Int J Cancer, 1992. 50(3): p. 373-81.

7. Chang, K., et al., Monoclonal antibody K1 reacts with epithelial mesothelioma but not with lung adenocarcinoma. Am J Surg Pathol, 1992. 16(3): p. 259-68.

8. Yamaguchi, N., et al., A novel cytokine exhibiting megakaryocyte potentiating activity from a human pancreatic tumor cell line HPC-Y5. J Biol Chem, 1994. 269(2): p. 805-8.

9. Bera, T. K. and I. Pastan, Mesothelin is not required for normal mouse development or reproduction. Mol Cell Biol, 2000. 20(8): p. 2902-6.

10. Rump, A., et al., Binding of ovarian cancer antigen CA125/MUC16 to mesothelin mediates cell adhesion. J Biol Chem, 2004. 279(10): p. 9190-8.

11. Gubbels, J. A., et al., Mesothelin-MUC16 binding is a high affinity, N-glycan dependent interaction that facilitates peritoneal metastasis of ovarian tumors. Mol Cancer, 2006. 5(1): p. 50.

12. Hassan, R., T. Bera, and I. Pastan, Mesothelin: a new target for immunotherapy. Clin Cancer Res, 2004. 10(12 Pt 1): p. 3937-42.

13. Ho, M., et al., Humoral immune response to mesothelin in mesothelioma and ovarian cancer patients. Clin Cancer Res, 2005. 11(10): p. 3814-20.

14. Yokokawa, J., et al., Identification of novel human CTL epitopes and their agonist epitopes of mesothelin. Clin Cancer Res, 2005. 11(17): p. 6342-51.

15. Thomas, A. M., et al., Mesothelin-specific CD8(+) T cell responses provide evidence of in vivo cross-priming by antigen-presenting cells in vaccinated pancreatic cancer patients. J Exp Med, 2004. 200(3): p. 297-306.

16. Hassan, R., et al., Tumor-directed radiation and the immunotoxin SS1P in the treatment of mesothelin-expressing tumor xenografts. Clin Cancer Res, 2006. 12(16): p. 4983-8.

17. Hassan, R., et al., Phase I study of SS1P, a recombinant anti-mesothelin immunotoxin given as a bolus I.V. infusion to patients with mesothelin-expressing mesothelioma, ovarian, and pancreatic cancers. Clin Cancer Res, 2007. 13(17): p. 5144-9.

18. Jaffee, E. M., et al., Novel allogeneic granulocyte-macrophage colony-stimulating factor-secreting tumor vaccine for pancreatic cancer: a phase I trial of safety and immune activation. J Clin Oncol, 2001. 19(1): p. 145-56.

19. Ellgaard, L. and E. M. Frickel, Calnexin, calreticulin, and ERp57: teammates in glycoprotein folding. Cell Biochem Biophys, 2003. 39(3): p. 223-47.

20. Chaput, N., et al., Molecular determinants of immunogenic cell death: surface exposure of calreticulin makes the difference. J Mol Med, 2007. 85(10): p. 1069-761

21. Obeid, M., et al., Leveraging the immune system during chemotherapy: moving calreticulin to the cell surface converts apoptotic death from “silent” to immunogenic. Cancer Res, 2007. 67(17): p. 7941-4.1

22. Obeid, M., et al., Calreticulin exposure dictates the immunogenicity of cancer cell death. Nat Med, 2007. 13(1): p. 54-61.

What is claimed is:

1. A method to produce a pancreatic antitumor composition effective in a patient comprising the steps of:
a. introducing into an isolated, non-tumorigenic cancer cell population a polynucleotide expression cassette having a functional a (1,3)-galactosyltransferase (αGT) protein;
b. isolating and enriching for a transduced cancer cell population which expresses αGal, mesothelin and/or carcinoembryonic antigen on the cell-surface; and
c. irradiating such cells.

2. An antitumor composition produced by the method of claim 1.

3. A method to produce a pancreatic antitumor composition effective in a patient comprising the steps of:
a. introducing into an isolated, cancer cell population a polynucleotide expression cassette having a functional α (1,3)-galactosyltransferase (αGT) sequence;
b. introducing into the cancer cell population of (a) a polynucleotide expression cassete having a mesothelin, calreticulin, and/or carcinoembryonic antigen sequence;
c. isolating a transduced cancer cell population which expresses αGal, mesothelin, calreticulin, and/or carcinoembryonic antigen on the cell-surface; and
d. irradiating such cells.

4. An antitumor composition produced by the method of claim 3.

5. An isolated, non-tumorigenic cancer cell population modified to express αGal, which also express mesothelin, calreticulin, and/or carcinoembryonic antigen (CEA) on the cell-surface, wherein after administration to a cancer patient, the production of antibodies to αGal, mesothelin, calreticulin, and/or carcinoembryonic antigen in said patient correlates with an improved overall survival.

6. The isolated cancer cell population of claim 5, wherein the cancer cell is a pancreatic cancer cell.

7. The isolated cancer cell population of claim 5, which induces a greater than a 10-fold increase in anti-αGal antibodies compared to baseline, wherein this increase correlates with improved overall survival.

8. The isolated cancer cell population of claim 5, which induces an increase in the levels of anti-mesothelin antibodies compared to baseline, wherein this increase correlates with improved overall survival.

9. The isolated cancer cell population of claim 8, wherein an increase of about 25% or more of anti-mesothelin antibodies compared to baseline correlates with improved overall survival.

10. The isolated cancer cell population of claim 5, which induces an increase in the levels of anti-carcinoembryonic antigen antibodies compared to baseline, wherein this increase correlates with improved overall survival.

11. The isolated cancer cell population of claim 5, wherein an increase in antibodies to one or more of αGal, mesothelin, calreticulin, and/or carcinoembryonic antigen in said patient correlates with an improved overall survival compared to the overall survival of patients exhibiting no increase in antibodies to these markers.

12. The isolated cancer cell population of claim 5, wherein an increase in antibodies to two or more of αGal, mesothelin, calreticulin, and/or carcinoembryonic antigen in said patient correlates with an improved overall survival compared to the overall survival of patients exhibiting an increase in antibodies to one of these markers.

13. The isolated cancer cell population of claim 5, wherein an increase in antibodies to αGal, mesothelin, calreticulin, and carcinoembryonic antigen in said patient correlates with an improved overall survival compared to the overall survival of patients exhibiting an increase in antibodies to two of these markers.

14. The isolated cancer cell population of claim 5, wherein said αGal expressed on the cell-surface is a trisaccharide of formula Galα1-3Galα1-4Glc, or Galα1-3Galα1-4GlcNAc.

15. The isolated cancer cell population of claim 5, which is administered in conjunction with one or more chemotherapeutic agents.

16. The isolated cancer cell population of claim 15, wherein the chemotherapeutic agent is gemcitabine.

17. The isolated cancer cell population of claim 5, which is administered in conjunction with radiation therapy.

18. The isolated cancer cell population of claim 13, wherein the radiation therapy is 5-FU radiation therapy.

19. The isolated cancer cell population of claim 5, which administered in conjunction with one or more chemotherapeutic agents and radiation therapy.

20. The isolated cancer cell population of claim 19, wherein the chemotherapeutic agent is gemcitabine and the radiation therapy is 5-FU radiation therapy.
